(19) United States
(12) Patent Application Publication
(10) Pub. No.: US 2015/0182617 A1
(43) Pub. Date: Jul. 2, 2015

(54) GLYCOPROTEINS FOR PSEUDOTYPING LENTIVECTORS

(71) Applicant: THERAVECTYS, Villejuif (FR)

(72) Inventors: Cecile Bauche, Paris (FR); Emeline Sarry, Malakoff (FR); Lucie Casaban, Malakoff (FR)

(73) Assignee: THERAVECTYS, Villejuif (FR)

(21) Appl. No.: 14/416,617

(22) PCT Filed: Jul. 25, 2013
(86) PCT No.: PCT/EP2013/065741

§ 371 Date: Jan. 22, 2015

Related U.S. Application Data
(60) Provisional application No. 61/675,441, filed on Jul. 25, 2012.

(51) Int. Cl. A61K 39/21; C12N 7/00; C12N 15/86

(52) U.S. Cl. CPC A61K 39/21; C12N 15/86; C12N 7/00; C12N 2740/16043; C12N 2760/00022

(57) ABSTRACT

The invention relates to compositions and methods based on the use of viral G proteins to pseudotype vectors. The viral G proteins can be expressed by expression vectors and can be used to pseudotype lentiviral vectors. The viral G proteins can be included in a combination of compounds for sequential administration to a mammalian host.

### CROSS-REFERENCE TO RELATED APPLICATIONS

This application claims the benefit of U.S. Provisional Application No. 61/675,441, filed Jul. 25, 2012, which is incorporated herein by reference.

### BACKGROUND OF THE INVENTION

The invention relates to the design of gene transfer vectors suitable for either a unique administration or for iterative administration in a host, and to their medicinal application. The field of application of the present invention concerns in particular animal treatment or treatment of human being (e.g. prophylactic or therapeutic or symptomatic or curative treatment) with lentivectors.

Recombinant vaccines have been developed with the progress of recombinant DNA technology, allowing the modification of viral genomes to produce modified viruses. In this manner, it has been possible to introduce genetic sequences into non-pathogenic viruses, so that they encode immunogenic proteins to be expressed in target cells upon transduction, in order to develop a specific immune response in their host.

Such vaccines constitute a major advance in vaccine technology (Kutzler et al., Nat Rev Genet, 9(10): 776-788, 2008). In particular, they have the advantage over traditional vaccines of avoiding live (attenuated) virus and eliminating risks associated with the manufacture of inactivated vaccines.

Gene delivery using modified retroviruses (retroviral vectors) was introduced in the early 1980s by Mann et al. (Cell, 33(1):153-9, 1983). The most commonly used oncogenic retroviral vectors are based on the Moloney murine leukemia virus (MLV). They have a simple genome from which the polyproteins Gag, Pol and Env are produced and are required in trans for viral replication (Breckpot et al., 2007, Gene Ther, 14(11):847-62; He et al. 2007, Expert Rev vaccines, 6(6):913-24). Sequences generally required in cis are the long terminal repeats (LTRs) and its vicinity: the inverted repeats (IR or att sites) required for integration, the packaging sequence ψ, the transport RNA-binding site (primer binding site, PBS), and some additional sequences involved in reverse transcription (the repeat R within the LTRs, and the polypurine tracts, PPT, necessary for plus strand initiation). To generate replication-defective retroviral vectors, the gag, pol, and env genes are generally entirely deleted and replaced with an expression cassette.

Retroviral vectors deriving from lentivirus genomes (i.e. lentiviral vectors/lentivectors) have emerged as promising tools for both gene therapy and immunotherapy purposes, because they exhibit several advantages over other viral systems. In particular, lentiviral vectors themselves are not toxic and, unlike other retroviruses, lentiviruses are capable of transducing non-dividing cells, in particular dendritic cells (He et al. 2007, Expert Rev vaccines, 6(6):913-24), allowing antigen presentation through the endogenous pathway.

Lentiviruses represent a genus of slow viruses of the Retroviridae family, which includes the human immunodeficiency viruses (HIV), the simian immunodeficiency virus (SIV), the equine infectious encephalitis virus (EIAV), the caprine arthritis encephalitis virus (CAEV), the bovine immunodeficiency virus (BIV) and the feline immunodeficiency virus (FIV). Lentiviruses can persist indefinitely in their hosts and replicate continuously at variable rates during the course of the lifelong infection. Persistent replication of the viruses in their hosts depends on their ability to circumvent host defenses.

The design of recombinant lentiviral vectors is based on the separation of the cis- and trans-acting sequences of the lentivirus. Efficient integration and replication in non-dividing cells is promoted by the presence of two cis-acting sequences in the lentiviral genome, the central polypurine tract (cPPT) and the central terminal sequence (CTS). These lead to the formation of a triple-stranded DNA structure called the DNA “flap”, which maximizes the efficiency of gene import into the nuclei of non-dividing cells, including dendritic cells (DCs) (Zennou et al., 2000, Cell, 101(2) 173-85; Arhel et al., 2007, EMBO J, 26(12):3025-37).

HIV-1 lentiviral vectors have been generated based on providing Gag, Pol, Tat and Rev proteins for packaging vectors in trans from a packaging construct (Naldini et al, PNAS 15: 11382-8 (1996); Zufferey et al, Nature Biotechnology 15:871-875, 1997); Dull et al, Journal of Virology (1997)).

Env proteins can also be provided in trans. Many viruses have envelopes that contain viral glycoproteins (viral G proteins). Glycoproteins contain oligosaccharide chains. The viral glycoproteins on the surface of the envelope assist with receptor binding and entry of the virus.

The glycoprotein of the vesicular stomatitis virus (VSV-G) is a transmembrane protein that functions as the surface coat of the wild type viral particles. It is also a common coat protein for engineered lentiviral vectors, replacing (“pseudotyping”) the wild-type lentiviral Env protein. The VSV-G protein presents an N-terminal ectodomain, a transmembrane region and a C-terminal cytoplasmic tail. It is exported to the cell surface via the transGolgi network (endoplasmic reticulum and Golgi apparatus). All VSV-G proteins do not achieve the same titers when used to pseudotype lentivectors. Charneau et al. (WO 2009/019612). For example, both Chandipura and Piry strains were shown to generate titers of less than 10⁵ IU/ml. Id.

Other envelope proteins that have been used for pseudotyping include those of lymphocytic choriomeningitis virus (LCMV), Ebola virus, Sindbis virus, Nipah virus, Ross River virus, Mokola, Rabies virus, Western Equine Encephalitis virus (WEEV), Venezuelan equine encephalitis virus (VEEV), Semliki Forest virus, and other retroviruses. The results for effective pseudotyping have been variable, and in many cases, the titers of pseudotyped lentiviral vectors have been low. For example, VEEV pseudotypes were reported to have a titer of 10⁶ IU/ml and a broad host range (Poluri et al., Journal of Virology, Vol. 82: 12580-12584, 2008). In contrast, Semliki Forest virus pseudotypes were reported to have lower titers (10³ to 10⁴ IU/ml), with those of Ross River virus pseudotypes at least 10 to 100-fold higher. Id. WEEV pseudotypes were reported to have titers less than 10⁵ IU/ml. Also, in many cases, the envelope proteins have required modifications to permit effective pseudotyping. Thus, to date, the production of high titer pseudotyped lentivectors has been unpredictable and difficult to achieve.

When using lentiviral vectors in vivo, the humoral response of the host elicited against the envelope protein used for pseudotyping the vector particles can interfere with subsequent administrations of the vector. The response which is elicited in the host against the envelope of the pseudotyped vector particles is accordingly a drawback for the efficient use of such vectors, when multiple administrations to the host are desired.

Charneau et al. (WO 2009/019612) showed that the antibodies against some VSV-G proteins could interfere with subsequent administrations of the vector using the same and other VSV-G proteins. Charneau et al. identified some iterative administrations regimens that could minimize this interference, but did not assess all combinations of VSV-G proteins.

Thus, there is a need in the art for compositions and methods to provide for effective pseudotyping and iterative administrations of lentiviral vectors. The present invention fulfills this need in the art.

### BRIEF SUMMARY OF THE INVENTION

The invention encompasses compositions and methods based on effective pseudotyping of lentiviral vectors with viral G proteins.

The invention encompasses a combination of compounds for sequential administration to a mammalian host comprising lentiviral vector particles, pseudotyped with a first viral G protein, for a first administration; and lentiviral vector particles, pseudotyped with a different viral G protein, for a second administration. The lentivector can be integrative or non-integrative.

The invention encompasses a combination of compounds for sequential administration to a mammalian host comprising (i) a composition comprising at least 10⁶ TU/ml of lentiviral vector particles pseudotyped with a first viral G protein, for a first administration; and (ii) a composition comprising at least 10⁶ TU/ml of lentiviral vector particles pseudotyped with a different, second viral G protein, for a second administration; wherein the first viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus G proteins.

The invention also encompasses a combination of compounds for sequential administration to a mammalian host comprising (i) lentiviral vector particles, pseudotyped with a first viral G protein, for a first administration; and (ii) lentiviral vector particles, pseudotyped with a different, second viral G protein, for a second administration; wherein the second viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus G proteins.

The first and/or second viral G protein can be selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus G proteins.

The first and/or second viral G protein can be selected from the VSV Indiana strain, the VSV New Jersey strain, the Cocal strain, the SVCV (Spring Viremia Carp Virus) strain, the Piry strain, or the Isfahan strain.

Preferably, the lentiviral vector particles for the first and/or second administration comprise a nucleic acid comprising a functional lentiviral DNA flap, most preferably, an HIV-1 DNA flap.

The invention encompasses a method for priming and subsequently boosting an immune response in a mammalian host comprising sequentially administering a combination of compounds of the invention, wherein the first administration and second administration are administered at different times to a mammalian host.

The invention encompasses a composition comprising at least 10⁶ TU/ml of unconcentrated lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus G proteins.

The invention encompasses a composition comprising at least 10⁸ TU/ml of concentrated lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Pity virus, Perinet virus, Snakehead rhabdovirus, Kimberley virus CS368, Jurona virus, West Caucasian Bat virus, Lagos bat 8619NGA, and Pike Fry rhabdovirus G proteins.

The invention encompasses an expression vector comprising a nucleotide sequence comprising a codon optimized G protein gene selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus G protein genes.

Preferably, the expression vector comprises a nucleotide sequence comprising the sequence of SEQ ID NO:1

The invention further encompasses uses of a viral G protein in the manufacture of a vaccine or medicament and for use in an iterative administration regimen. The invention further encompasses methods and uses for priming and subsequently boosting an immune response in a mammalian host comprising sequentially administering the combination of compounds of the invention, wherein the first administration and second administration are administered at different times to a mammalian host.

### BRIEF DESCRIPTION OF THE DRAWINGS

The invention is better understood in reference to the drawings, in which

FIG. 1 depicts production yields with different pseudotyping VSV-G proteins.

FIG. 2 depicts the effect of WPREm on production yields with different pseudotyping VSV-G proteins.

FIG. 3 depicts sero-neutralization of different pseudotyping VSV-G proteins with different sera. ++: Neutralization more than 90%. +: Neutralization between 70 and 90%. +/−: Neutralization between 50 and 70%. Experiments performed with rabbit sera instead of mouse sera are shaded in gray.

FIG. 4 depicts specific T-cell responses with different pseudotyping VSV-G proteins.

FIG. 5 depicts titers obtained after pseudotyping lentiviral vectors with various viral G proteins. A lentiviral vector expressing the GFP under the control of a CMV promoter was pseudotyped with various viral G proteins (codon optimized for human use). Titers of bulk productions were then evaluated by FACS.

FIG. 6 depicts titers obtained after pseudotyping lentiviral vectors with various viral G proteins, after ultracentrifugation. Some of the more efficient viral G proteins were evaluated for their ability to pseudotype the GFP coding lentiviral vector. For each envelope, a fraction of the bulk productions was evaluated by FACS and the remained fraction concentrated by ultracentrifugation and evaluated by qPCR.

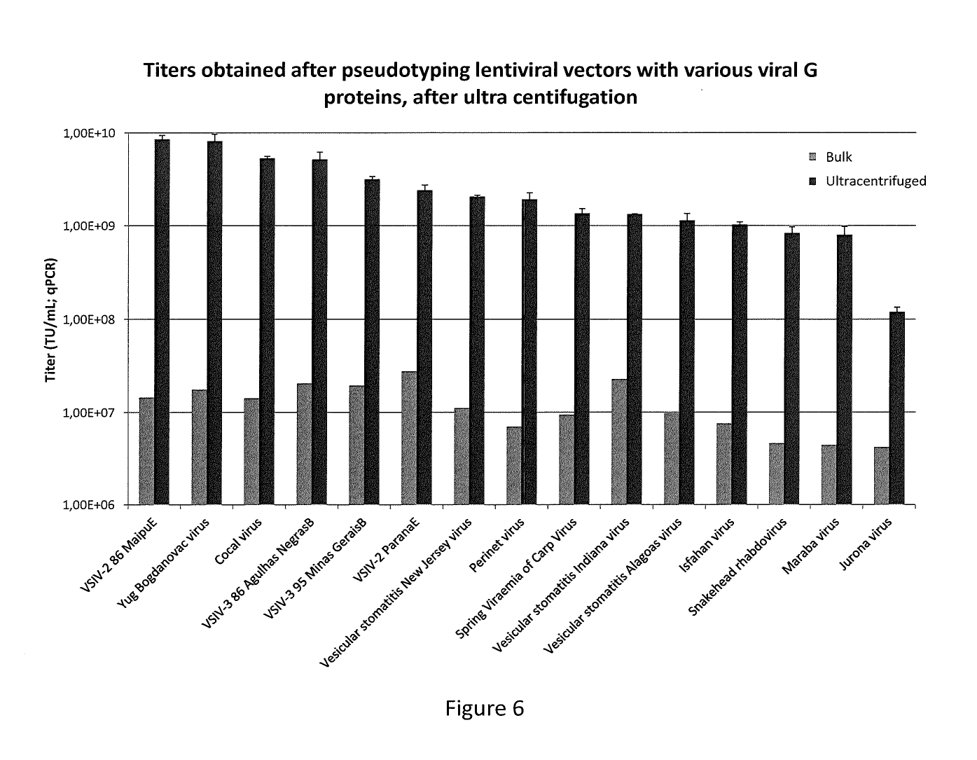

### DETAILED DESCRIPTION OF THE INVENTION

The glycoprotein of the vesicular stomatitis virus (VSV-G) is a transmembrane protein that functions as the surface coat of the wild type viral particles. Some VSV-G proteins have been used for engineering lentiviral vectors. The suitability of various additional VSV-G proteins and other viral G proteins to pseudotype lentivectors was evaluated.

First, codon optimized genes of VSV Alagoas and viral Hemoragic septicemia virus FR-L59X G proteins were synthesized, and cloned between the BamH1 and EcoR1 sites of the pThV-plasmid, with or without the WPREm sequence, generating the pThV-VSV.G(ALAGOAS-CO), pThV-VSV.G(ALAGOAS-CO)-WPREm, pThV-VSV.G(FR-L59X-CO) and pThV-VSV.G (FR-L59X-CO)-WPREm vectors.

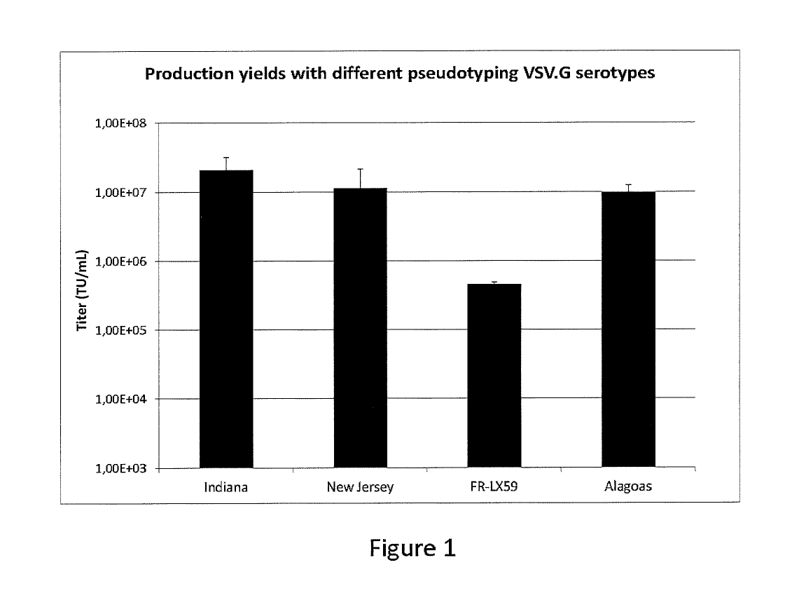

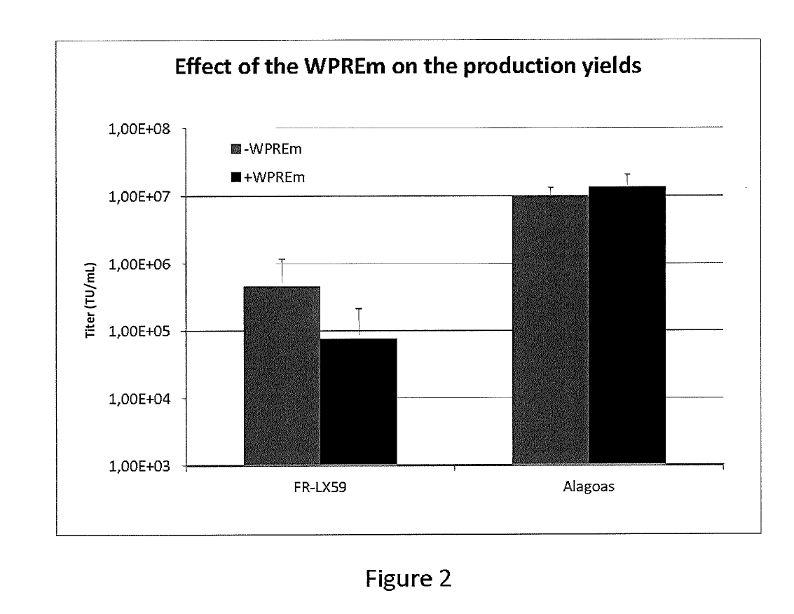

The ability of these viral G proteins to pseudotype lentivectors was evaluated for lentiviral particle production by determining the vector titers (TU/ml) after cotransfection. As shown in FIG. 1, both VSV Alagoas and viral Hemoragic septicemia virus FR-L59X G proteins could pseudodype lentiviral particles. The codon optimized Alagoas VSV-G protein resulted in a titer of approximately 10⁷ TU/ml. In contrast, the codon optimized viral Hemoragic septicemia virus FR-L59X G protein resulted in much lower production yields (less than 10⁶ TU/ml). Furthermore, it appeared that the addition of the WPREm did not increase the lentiviral production yields (FIG. 2).

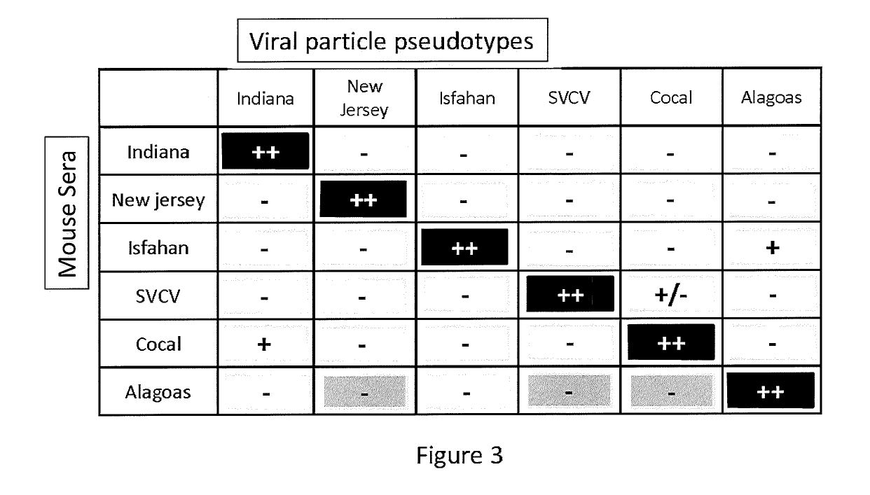

The ability of Alagoas VSV-G protein to generate neutralizing antibodies was assessed. Sera from mice injected with lentivectors pseudotyped with various VSV-G proteins (Indiana, New Jersey, Cocal, Isfahan, SVCV and Alagoas) were assessed for their ability to neutralize viral particles pseudotyped with various viral G proteins. As shown in FIG. 3, lentiviral vectors pseudotyped by the Alagoas G protein are not neutralized by mouse sera generated by Indiana, New Jersey, Cocal and SVCV G proteins. On the other hand, Isfahan antibodies do neutralize lentiviral vectors pseudotyped by the Alagoas G protein. Furthermore, serum from animals injected with lentivectors pseudotyped with Alagoas VSV-G protein did not neutralize viral particles pseudotyped with VSV-G proteins (Indiana, New Jersey, Isfahan, SVCV and Cocal), other than Alagoas. These data indicate that in an iterative immunization regimen, Isfahan pseudotyped lentivectors should not be administered before (but only after) lentivectors pseudotyped with Alagoas to avoid any neutralizing reaction.

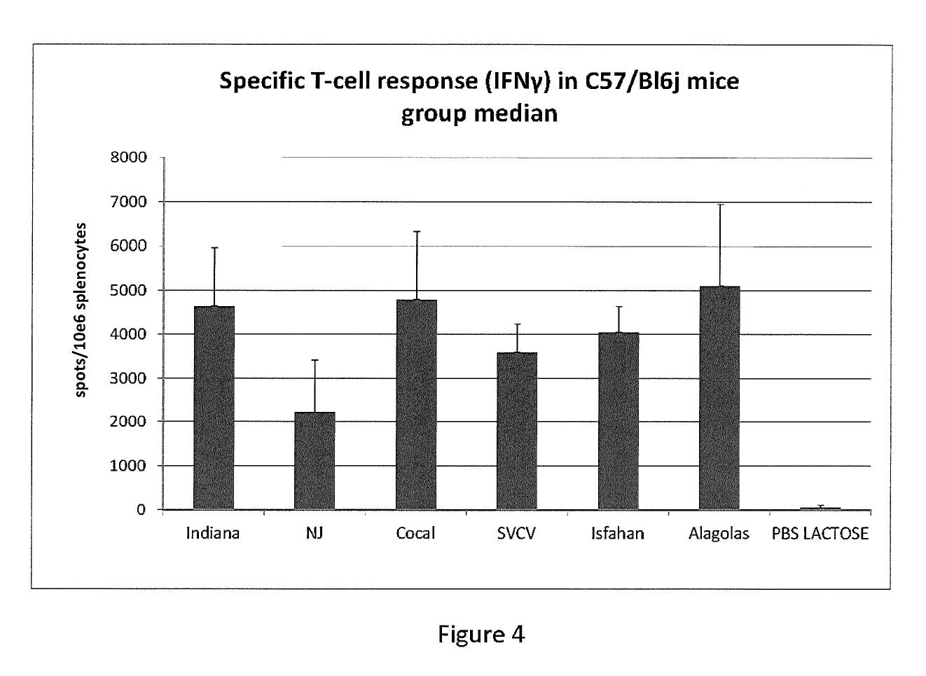

To assess immune responses generated by pseudotyped lentivectors, mice were immunized with lentivectors (10⁶ TU) encompassing an HIV antigen, and pseudotyped with various viral G proteins (Indiana, New Jersey, Cocal, SVCV (Spring Viremia Carp Virus), Isfahan and Alagoas. 12 days after immunization, the specific T-cell responses were monitored in mice splenocytes by IFN-γ ELISPOT. As shown in FIG. 4, all lentivectors elicit T cell responses of similar ranges. The lentivectors also showed T cell responses of similar epitope coverage.

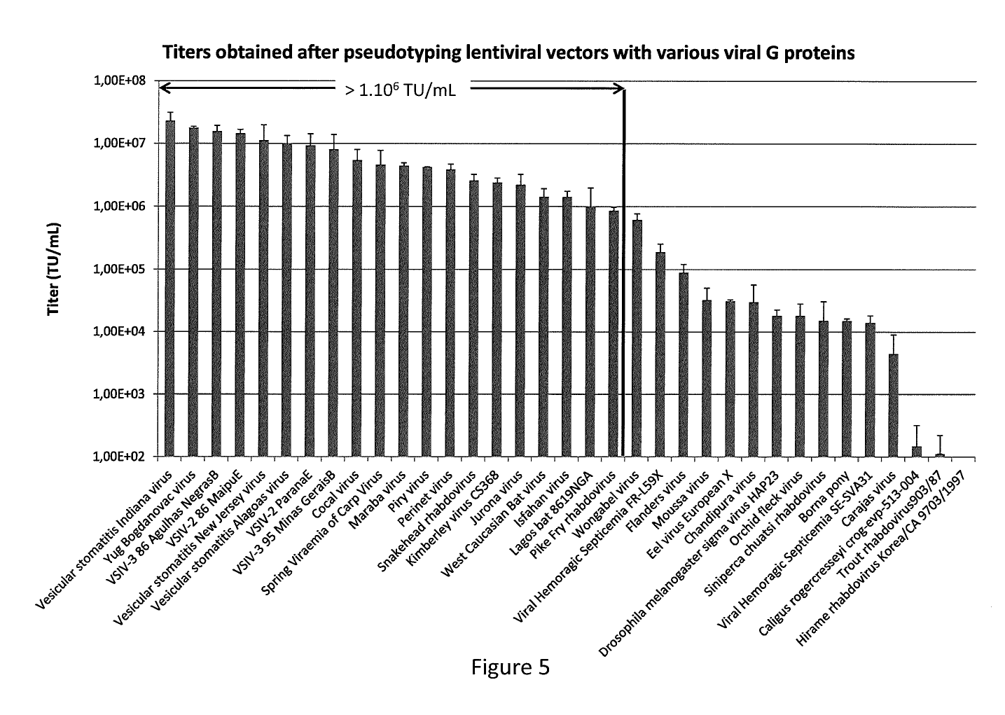

Next, the ability of other viral G proteins to pseudotype lentivectors was evaluated for lentiviral particles production by determining the vector titers (TU/ml) after cotransfection. The other viral G proteins included those from Vesicular stomatitis Indiana virus, Yug Bogdanovac virus, VSIV-3 86 Agulhas NegrasB, VSIV-2 86 MaipuE, Vesicular stomatitis New Jersey virus, Vesicular stomatitis Alagoas virus, VSIV-2 98 ParanaE, VSIV-3 95 Minas GeraisB, Cocal virus, Spring Viraemia of Carp Virus, Maraba virus, Piry virus, Perinet virus, Snakehead rhabdovirus, Kimberley virus CS368, Jurona virus, West Caucasian Bat virus, Isfahan virus, Lagos bat 8619NGA, Pike Fry rhabdovirus, Wongabel virus, viral Hemoragic septicemia virus FR-L59X, Flanders virus, Moussa virus, Eel virus European X, Chandipura virus, Drosophila melanogaster sigma virus HAP23, Orchid fleck virus, Siniperca chuatsi rhabdovirus, Viral Hemoragic Septicemia SE-SVA31, Carajas virus, Caligus rogercresseyi crog-evp-513-004, Trout rhabdovirus 903/87, and Hirame rhabdovirus Korea/CA 9703/1997. The results are shown in FIG. 5.

As can be seen in FIG. 5, many of the viral G proteins provided poor titers. However, viral G proteins from Vesicular stomatitis Indiana virus, Yug Bogdanovac virus, VSIV-3 86 Agulhas NegrasB, VSIV-2 86 MaipuE, Vesicular stomatitis New Jersey virus, Vesicular stomatitis Alagoas virus, VSIV-2 98 ParanaE, VSIV-3 95 Minas GeraisB, Cocal virus, Spring Viraemia of Carp Virus, Maraba virus, Piry virus, Perinet virus, Snakehead rhabdovirus, Kimberley virus CS368, Jurona virus, West Caucasian Bat virus, Isfahan virus, Lagos bat 8619NGA, Pike Fry rhabdovirus, Wongabel virus, viral Hemoragic septicemia virus FR-L59X, and Flanders virus provided titers of greater than 10⁵ TU/ml, with most of these greater than 10⁶ TU/ml, and some of them greater than 10⁷ TU/ml. The G protein of Piry virus used in these experiments had the amino acid sequence of SEQ ID NO:30. This amino acid sequence differs from the amino acid sequence of the G protein of Piry virus previously used for pseudotyping, which was shown to be quite inefficient (Charneau et al. WO 2009/019612). The amino acid sequence of each of these viral G proteins is shown below, except for Vesicular stomatitis Indiana virus (GenBank: CAC47944.1), Vesicular stomatitis New Jersey virus (UniProtKB/Swiss-Prot: P04882.1), Cocal virus (UniProtKB/Swiss-Prot: 056677.1), Spring Viraemia of Carp Virus (GenBank: AAZ20272.1), and Isfahan virus (NCBI Reference Sequence: YP_{—}007641385.1), which have their published sequences.

YUG BOGDANOVAC VIRUS GenBank: AFH89679.1:

(SEQ ID NO: 5)
MISSTLILVIISAHAFCDMIIPFPDVTTTSWKPVLRGEHHCPASNDLDMA
GGLSTLKMNVKIPSGVVGSKSDGYLCHGAKWVTTCDYRWYGAKYITHSLH
PLRPSTSQCFDAIKAYREGTLLSPGFPPESCGWNSVTDSELLSIQITPHH
SGVDDYRGVWIDSMFPKGECDQRICDTVQEHSIWIAANNVSSACSIAFKQ
LEGYFYYRNSGIQPNKDGTFFHSSHHPNSPMSSCCRIKYCNQEGLRLHTG
EWIGVAWNTKIRDVTLDSYTDTCPGGTEVKSTIGSSPTRVVAWEMERIMD
FALCQNVWDKVNRGEQLSPLDLSYLSSRAPGKGLAYTIINETLHVAHVRY
IRTWIKGPVLKEIKGRRGSSSAAEDTLWIQWFPFGDNQIGPNGLLKSNGT
FKFPFYLVGVGALDEDLIEMANADPVDHLQRVDAETHMRGDEELFFGDTG
VSKNPIESVEGWFSNWISGLFNISIIVLCVLSVLIVFKSVITLIRVVRRR
RRPRAEEDVELNNMNPRPQTRQPVGAPNIIPGAWGIQPSHGRGVRQSQFV
KRSALNIVT.

WONGABEL VIRUS NCBI Reference Sequence: YP_{—}002333278.1:

(SEQ ID NO: 6)
MSFLLAIIIFFFRTQDSNGYVVQSGIGGIRESKVNILGQVVPYSTKFKHS
VLKSQWDYRHDKGHRTVLPTNCHATWNDITPSNMRCPRRKIIGKDGLYNT
YIGDFWHPHTDKGSEVKGFICQKTKVVVSTCIETWYFSTTKETQIDEVPI
TKEDCLAAITLVDSGEYIEPFFPPHVCSWASTNKNSKEFVTVHEHSVVLD
IYENKLMDPIFLAGKCFDKVCKTIHRNVLWVEANDNERDDFCVATAWEYS
HVFADIDIDHNNNHPIYSIGKTIDSEIYGPRDLADACIIKICGIPGIRFS
HGEWWGIKTLSDRIPLEDIIIKCHNGTSVGFVHNIWTPSELVGEITYRDH
KCLDVLSSFLGQRKINPYELSYLVQDFPGEGPAYRIMKQYTGNNKTKATF
RLQTKTCRYHVAYIDKLTFDPENGTDEVYKLGTWGNGRTVILNSTEVGIN
PTYINKSFDWEPLETFNGLMRFGAELVLPQAVYTDHPNITNLLEDYEISL
IGHPKEIFEPEQDELSQVYKFYERSNSTNVVGLASNFVKTIGRSIGNFFG
GTKNLIWWLVTVALSTIGTYIAYKLGLFKFLGRILFQGSESKEDKRVSNI
YEEPLKLGGRRSHLVKNPFFDNGI.

MOUSSA VIRUS ISOLATE C23 GenBank: ACZ81401.1:

(SEQ ID NO: 7)
MRTLVIWFLINVTMAFAKPPGSASLSLGLYWVPRIDNNTWKSVHTTNLVC
PSFVGSVLPEMEESFEIDIQVPKHSQTTSHQGGYLCYGFSFSVVCEEGFW
GGQKVTEHTFTHLVSSEECLKAIEDKKSGEYRPPHTPVSECGWMQTNTKT
LRFVTLEEHPVLFDPYTVNFVDGLFEKTLCNQRICPTVHANTIWIGDNEP
KKDCPPTENEKAVLYVEKQNWPVVWVKLTGGTVYKLDRACTMTYCDIDGV
RMEDGHWFAGVNLTQYVRRDCDKGMDITFDTLASLSLLTKIELEHVQDRM
ECLDAVQDLRAGGKVTYAKLSKLQPKRGGLFHVYRINKGTLEYTMGRYEG
LTSLITNIPFVIGKNQKDEKVQLHHVPSGDNSTLSSYNGVHMFLNGTVII
PEMELYKLRYSETLLYEHLLGKMKHPSAKQRERMGLTPDDDKRTTNKSLN
IGEWFSSFWSHLVGKIVSILGTALAIFLILYICWTCLKIQIKRVSDKNRV
DQMEMQILSKARAPEVRPTLSGPIW.

WEST CAUCASIAN BAT VIRUS UniProtKB/Swiss-Prot: Q5VKN9.1:

(SEQ ID NO: 8)
MASYFALVLNGISMVFSQGLFPLYTIPDHLGPWTPIDLSHLHCPNNLYTD
ASYCTTEQSITYTELKVGSSVSQKIPGFTCTGVRTESVTYTNFVGYVTTT
FKKKHFPPKSRDCREAYERKKAGDPRYEESLAHPYPDNSWLRTVTTTKDS
WVIIEPSVVELDIYTSALYSPLFKDGTCSKSRTYSPYCPTNHDFTIWMPE
SENIRSACNLFSTSRGKLVRNRTSTCGIIDERGLFRSVKGACKISICGRQ
GIRLVDGTWMSFRYSEYLPVCSPSQLINTHDIKVDELENAIVLDLIRRRE
ECLDTLETILMSGSVSHRRLSHFRKLVPGSGKAYSYINGTLMESDAHYIK
VENWSEVIPHKGCLMVGGKCYEPVNDVYFNGIIRDSNNQILIPEMQSSLL
REHVDLLKANIVPFRHPMLLRSFTSDTEEDIVEFVNPHLQDTQKLVSDMD
LGLSDWKRYLLIGSLAVGGVVAILFIGTCCLRCRAGRNRRTIRSNHRSLS
HDVVFHKDKDKVITSWESYKGQTAQ.

KIMBERLEY VIRUS ISOLATE CS368 GenBank: AFR67091.1:

(SEQ ID NO: 9)
MSSEIKMLIELMLFSILSCVISQRVYNFPFNCTEPERIKDYQIKCPIRQN
EVSLEAHHVEVDEKIEKICRPQIKDDDHIEGYICREQHWTTKCTETWYFS
TEIEYTIKETVPNQADCQKELEKLKRGISIPPYYPPAGCFWNMAQSEKIT
FWLVPHKVLQNPYDMKLYDPGFIEKCDVKKAKTKGCKMKDITGLWVTNND
GKNTSEHCNKDHWECIGIKSFRSELNLHDRLWESPELGIMKLNKACKKNF
CGYKGVILEDGEWWGYTNVADSEIEYVHLNNCDDSRLPGFRIHQDRTEFE
EFDIKAEMENERCMNTLSKILNKENLNFVDMSYLSPSRPGRDYAYLFEQV
SWDETFCLTWPDSRKSKNCKVDWKVHKNAGLVTKKHGAIGTYYRSMCMYY
PIEDTNKDGILQKDELKDKGIPGKNRYRTLKRSKNDYGEDSEFNITYNGM
VVVNESFHMAVKSIYDGTEDYNSLLKFEVSEFDKIDLNEAYKEEENKWND
IDLTPVSSVNRSRSDIIKEVEKGGRKIISAVTGWFTGLAKTVRWTIWGIG
SIVTIYAIWKLKKMITKKNKEDKNLVNHNELNEAFEMSKDVERGRVETWI
RKNKGKEEGIYEQVSDIEDNVSKYERGVHASKGGDKMNVYSPHGKNGKKG
FFNH.

FLANDERS VIRUS GenBank: AAN73287.1:

(SEQ ID NO: 10)
MSYLIVKWILFLVGIDKQVLSWTHDSGRSFVRQYHDPNWFDQTMVYPIEC
NSTWQEVNTLNLRCPKSLKIDPKNKLNFDLGTVYHPLPSSRYVVNGYICH
KQTWISKCEETWYFSTTETNKIENVPITPEDCREAVTIYEMGEYVNPFFP
PFYCSWSSTQIDKKTFVIVEPHIVKEDIYNKTFIDPFFLNGYCDQLPCKT
IHPDVLWVPQELQKRKDLCNKGTWETGKVFGVLEEKLYQNGYLKDNRFGI
DEQWIRSSIYGLRSLVGSCYRGVCRQFGIRFKTGEWWGLEGKDVTGWIKQ
IIPRCQENQYVSFHHDNSDENIAEAQLVARELVCEEFLGRAKGGDLISPF
DLNYLLPLNPGLGPSYRAFKRILKKDSHGGSSPQFRLEKRDCIYSVVHNV
TEKVNITNNKLAIGQLFDGSYVYINESEFSRPDYLNNSDNASRDDWFLLS
LNGMIKYGNSVYLPHGVSTGLSGIQDIVERGTLMLLDHPKSIAISNQMDL
AKNIYTSYFQMNTTSIGSKIENMIIRAKNAVSSYFSQLTNIAWWIGTGIL
GLLGFIVIKRFHLIQLICGKKHRNGKIKKKNNKLNNDDQEAHVYDTIFNT
PKTPPHGKGTGVKYFDY.

VESICULAR STOMATITIS VIRUS ISOLATE VSIV-2 86MAIPUE GenBank: AEM60931.1:

(SEQ ID NO: 11)
MNFILLILAVLPISNYAKFSIVFPQSQKGNWKNVPASYHYCPSSSDQNWH
NDLIGITMKVKMPKTHKAIQADGWMCHAAKWITTCDFRWYGPKYITHSIH
SIQPTSEQCKESIKQTKQGTWISPGFPPQNCGYATVTDSVAVIVQATPHH
VLVDEYTGEWIDSQFPNGKCEAEECETIHNSTVWFSDYKVTGLCDATLVD
TDITFFSEDGKKESLGKPNTGYRSNYFAYEKGEKVCKMNYCKHSGVRLPS
GVWFEFADQDAYTAAKFPECPVGATISAPTQTSVDVSLILDVERILDYSL
CQETWSKIRSKQPVSPVDLSYLAPKNPGTGPAFTIINGTLKYFETRYIRI
DIDNPIIPKMVGKIGGSQTERELWTEVVYPYEDVEIGPNGILKTPTGYKF
PLFMIGHGMLDSDLHKTSQAEVFEHPHLAEAPKQLPEEETLFFGDTGISK
NPVELIEGWFSSWKSTVVTFFFAIGVFILLYVIARIVITVRSRYQGASNK
RIYNDIEMSRFRK.

VESICULAR STOMATITIS VIRUS ISOLATE VSIV-3 86AGULHAS NEGRASB GenBank: AEM60936.1:

(SEQ ID NO: 12)
MNPAFVLCMLLAGSSWAKFAIVFPQSQKGEWKDVPPNYRYCPSSADQNWH
GDLLGVNIRAKIPKAHKAIKADGWMCHAAKWITTCDYRWYGPQYITHSIH
SFVPTQAQCEESIKQTKEGVWINPGFPPKNCGYASISDAESVIVQATAHT
VMIDEYSGDWLDSQFPTGRCKETICETIHNSTLWYADYKVTGLCDSALVS
TEVTFYSEDGLVTSIGRQNTGYRSNYFPYEKGAAACRMKYCTYDGIRLPS
GVWFEMVDKELLDSAQMPECQAGLTISAPTQTSVDVSLILDVERMLDYSL
CQETWSKVHNGQPISPVDLGYIAPKNPGAGPAFTIINGTLKYFDTRYLRI
DIDGPVLKKMAGKISGTLTERELWTEWFPYEDVEIGPNGVLKTPEGYKFP
LYMIGHGLLDADLQKTSQAEVFHHPQIAEAIKKLPDDETLFFGETGISKN
PVELIEGWFSNWRSSVMAVVFAIALLVVLVLIVRIIVAFRHSCCRRQRKI
YDDLEMNQLRK.

MARABA VIRUS GenBank: AEM60927.1:

(SEQ ID NO: 13)
MLRLFLFCFLALGAHSKFTIVFPHHQKGNWKNVPSTYHYCPSSSDQNWHN
DLTGVSLHVKIPKSHKAIQADGWMCHAAKWVTTCDFRWYGPKYITHSIHS
MSPTLEQCKTSIEQTKQGVVVINPGFPPQSCGYATVTDAEVVVVQATPHH
VLVDEYTGEWIDSQLVGGKCSKEVCQTVHNSTVWHADYKITGLCESNLAS
VDITFFSEDGQKTSLGKPNTGFRSNYFAYESGEKACRMQYCTQWGIRLPS
GVWFELVDKDLFQAAKLPECPRGSSISAPSQTSVDVSLIQDVERILDYSL
CQETWSKIRAKLPVSPVDLSYLAPKNPGSGPAFTIINGTLKYFETRYIRV
DISNPIIPHMVGTMSGTTTERELWNDWYPYEDVEIGPNGVLKTPTGFKFP
LYMIGHGMLDSDLHKSSQAQVFEHPHAKDAASQLPDDETLFFGDTGLSKN
PVELVEGWFSSWKSTLASFFLIIGLGVALIFIIRIIVAIRYKYKGRKTQK
IYNDVEMSRLGNK.

VSIV-2 98 PARANAE GenBank: AEM60932.1:

(SEQ ID NO: 14)
MNFLLLFLVTIPAYSYAKFSIVFPQSQKGNWKNVPATYHYCPSSSDQNWH
NDLIGIPMKVKMPKTHKAIQADGWMCHAAKWITTCDFRWYGPKYITHSIH
SIQPTSEQCKESIKQTKQGTWINPGFPPQNCGYASVSDSVAVIVQATPHH
VLVDEYTGEWIDSQFPNGKCKAEECETIHNSTVWFSDYKVTGLCDATLVD
TDITFFSEDGKKESLGKPNTGYRSNYFAYEKGEKVCKMNYCKHAGVRLPS
GVWFEFADQDAYTAAKFPECPVGATISAPTQTSVDVSLILDVERILDYSL
CQETWSKIRSKQPVSPVDLSYLAPKNPGTGPAFTVINGTLKYFETRYIRI
DIDNPIIPKMVGKVGGSHTERELWTEWYPYEDVEIGPNGILKTPTGYKFP
LFMIGHGMLDSDLHKTSQAEVFEHPHLAEAPKQLPEEETLFFGDTGISKN
PVELIEGWFSSWKSTVVTFFFAIGVFILFCVVARIVIAVRYRYQGASKKR
IYNDVEMSRFRK.

VSIV-3 95 MINAS GERAISB GenBank: AEM60937.1:

(SEQ ID NO: 15)
MNLAVVLCMLLAGSSWAKFTIVFPQSQKGEWRDVPSNYRYCPSSADQSWH
GDLLGVNIRAKIPKAHKAIKADGWMCHAAKWITTCDYRWYGPQYITHSIH
SFVPTQAQCEESIKQTKEGVWINPGFPPKNCGYASINDAESVIVQATAHT
VMIDEYSGDWLDSQFPTGRCKGTICETIHNSTLWHADYKVTGLCDAALVS
TEVTFYSEDGLVTSIGRQNTGYRSNYFPYEKGAGACRMKYCTHDGIRLPS
GVWFEMVDKELLNSSQMPECPAGLTITAPTQTSVDVSLILDVERMLDYSL
CQETWSKVHNGQPISPVDLGYIAPKNPGAGPAFTIINGTLKYCDTRYLRI
DIDGPVLQKMTGKVSGTPTERELWTEWFPYEDVEIGPNGVLRTQEGYKFP
LFMIGHGLLDADLQKTSRAEVFHHPQIAEAVKKLPDDETLFFGETGISKN
PVELIEGWFSNWRSSVMAVIFAIVSLVLLGLIVRVIVAFRNSCCQRKRKV
YNDLEMNRLRN.

JURONA VIRUS GenBank: AEG25348.1:

(SEQ ID NO: 16)
MESLPFSALLAVLSITLCDSAIPIFFPSEPQLEWKPVLPGSRYCPQSNEM
SLDPDLKKSTISVKVPIGVTPSKSDGYLCHGAKWVSTCDFRWYGPKYITH
SIHNLRPTTNDCEDAIKKYEAGTLINPGFPPDSCAYATVTDSEHLVILIT
PHHVGVDDYRGAWVDDSFPSGVCETNQCDTTHNSSIWIPKTKTRHNICSQ
TFANLSVTISYREGGAMKGADMVFHSKYHPHMVGGHICKMNFCNKQGLRL
QNEEWIEIPSGTKVGNQDLMNLFSDCKSGLEVRSTLRSEGANTLTWETQR
LLDYALCQNTWDKFDNQGAVSALDLSYLAARAPGKGVAYTMINGTLHSAP
TRYVRMWIESPSMEELKAKKESSSGVETSIWNQWFPFKGGEIGPNGLIKA
GNKYKFPLYLVGMGMLDDEINALELGGPIDHPQRAHAQAVLGDEETLFFG
DTGVGKNPVELITGWFSGWKETIMAVVAIFLLVIVLYGVLRCCPTICVLC
KRKSRHRTKDMEMQYIPNNQRHWR.

PERINET VIRUS GenBank: AEG25354.1:

(SEQ ID NO: 17)
MSSKIVLAAICLCSVQYVACSFQIVFPEFNNAAWLPYLKTSRYCPQSAEM
EFERRVSTTLLSADVPIGVTPTKSDGYLCHAAKWVTTCDFRWYGPKYVTH
SIHDLTPAQVDCHEALARYKAGTLFNPGFPPASCGYATITDSEQKVVMIT
PHHVGIDDYRGKWIDPIFPGGECTTNYCETLHNSSVWLPADEKIVDICAQ
TFRKIKVTATYPSEGAVTKETISLHSAYHPHVPGTGICRMTYCSKEGLRL
PNGEWLGIFYDNRIKTTDVRTVFPACPDGLEVKSTLNSDGANTIAWETQR
MLDYALCQSTWDKVQNKEPLSAVDLSYLSARSPGKGLAYTVINGTLHFAH
VRYVRTWIDGPVLKDLKGSRFDPTAAQKTLWDQWFPFGSNEIGPNGLLKT
PKDFKFPLYIIGTGLVDEDLQELSEAGPIDHPQIPDASGILPNSEQVYYG
DTGVSKNPIELIEGWFANWKETVMSIVGLVLLITIVFTVLKCIGTCRSLR
RKRKIEKDIELQEIGPYQPTTYRPR.

EEL VIRUS EUROPEAN X GenBank: CBH20129.1:

(SEQ ID NO: 18)
MDTLIKILLIIVILKSLHAHIEFVPHDLSKWRDISIEHLDCPIYGDLSNQ
ATETTPVKYSSVQWGLKNNIDGYLCISAKWSVTCDYRWYGSKYISTSIEY
VPTKESECRDAIKSSKNGELVSPHFMPENCGWNNVLTESVTYTTVSSHEV
KLDPYQMTFVDSLFPGGKCSSSVCSTIYHQGVWINPSNNLGFCKDPVDHQ
GQLYMAGLVGARGEIVKEVWNLRSVFKPEIGRSKHLTGSCWMTYCDQRGL
RFSDGEWAGFQIPEISALKQVLLGLPECKDDVLVHAHDTNTELREILEHM
DESALNAICQQEVRRAKERGWSDWLLSMMTPFTEGLGPVYRLNKGKLEAS
MGYYRKVYIDSSNAPQAFGQTEDKESVGWSDLVPKDADGAILSMYNGNVV
INNQIKWAKNALGSHILDEISALEFETPVVHHPHLTILSVNHSDLVSSTH
PNGQGVNLIESVSHWAGGLWASIGSGLMILVLVALVGFCTIKVCLAYVPS
IWAQKNRNGKRRGTTSQRSTEQEMFELSAV.

LAGOS BAT VIRUS UniProtKB/Swiss-Prot: Q8BDV6.1:

(SEQ ID NO: 19)
MSQLNLIPFFCVIIVLSVEDFPLYTIPEKIGPWTPIDLIHLSCPNNLQSE
DEGCGTSSVFSYVELKTGYLTHQKVSGFTCTGVVNEAVTYTNFVGYVTTT
FKRKHFKPTALACRDAYHWKISGDPRYEESLHTPYPDNSWLRTVTTTKES
LVIISPSIVEMDVYSRTLHSPMFPTGTCSRFYPSSPSCATNHDYTLWLPD
DPNLSLACDIFVTSTGKKSMNGSRMCGFTDERGYYRTIKGACKLTLCGKP
GLRLFDGTWISFPRPEVTTRCLPNQLVNIHNNRIDEVEHLIVEDLIRKRE
ECLDTLETVLMSKSISFRRLSHFRKLVPGYGKAYTILNGSLMETNVHYLK
VDNWSEILPSKGCLKINNQCVAHYKGVFFNGIIKGPDGHILIPEMQSSLL
KQHMDLLKAAVFPLKHPLIEPGSLFNKDGDADEFVDVHMPDVHKLVSDVD
LGLPDWSLYALIGATIIAFFILICLIRICCKKGGRRNSPTNRPDLPIGLS
TTPQPKSKVISSWESYKGTSNV.

SNAKEHEAD RHABDOVIRUS NCBI Reference Sequence: NP_{—}050583.1:

(SEQ ID NO: 20)
MTLPNMKPKRIVLFLVFLNAWVSNAQVTHKPRPDSIVEYSEEWENPIYTT
PSHCFEDTFAPVKPEKLRCPHIFDDQNLGLTASKAKILHMDLKPEDTHFE
AKGRLLHKVTYQVLCSTGFFGGRTVTRKVLETPMGDNEAQAYKAVDREFP
YFPEPLCFWLRDNVAAERVFHFSTPKTVTVDLYSRKYISPDFVGGQCAKS
PCPTHWPNVYWVGETQSPECPSIDTEGGHIFTKKDTHRITKAVVHGHHPW
GLTKACQIQFCNEQWIRTDLGDLIRIEPNDGTSSLTLPKCQDNVVQMRGN
LDDFSYLNHAIVNMAQRSECLEAHSSIVAQQKVSPYLLSKFRPPHPGLGK
AHYLQNNTIMRGDCIYEGVAEISENRTTYRNLKGEWKKWSLSRGGEGYDG
MTVGTKIVIPDLEKYQSIYDNGMFIPKLLGEVPHPSIVITYNQTDSIETG
IFTDGKLLNMGVNWTLWPSLSGISLFTVASLILIWYCCCRVTPQALNYSI
PMHTITSRGVEI.

VIRAL HEMORAGIC SEPTICEMIA SE-SVA31 GenBank: ACH89137.1:

(SEQ ID NO: 21)
MEWNTFFLVILIIIIKNTTPQITQRPPVENISTYHADWDTPLYTHPSNCR
EDSFVPIRPAQLRCPHEFEDINKGLVSVPTRIIHLPLSVTSVSAVASGHY
LHRVTYRVTCSTSFFGGQTIEKTILEAKLSRQEATNEASKDHEYPFFPEP
SCIWMKNNVHKDITHYYKTPKTVSVDLYSRKFLNPDFIEGVCTTSPCQTH
WQGVYWVGATPTAHCPTSETLEGHLFTRTHDHRVVKAIVAGHHPWGLTMA
CTVTFCGTEWIKTDLGDLIQVTGPGGARKLTPKKCVNTDIQMRGATDDFS
YLNHLITNMAQRTECLDAHSDITASGKISSFLLSKFRPSHPGPGKAHYLL
DGQIMRGDCDYEAVVSINYNSAQYKTVNNTWKSWKRVNNNTDGYDGMIFG
DKLIIPDIEKYQSVYDSGMLVQRNLVEVPHLSIVFVSNTSDLSTNHIHTN
LIPSDWSFNWSLWPSLSGMGVVGGAFLLLVLCCCCKASPPIPNYGIPMQQ
FSRSQMV.

CARAJAS VIRUS FW339542.1:

(SEQ ID NO: 22)
MKMKMVIAGLILCIGILPAIGKITISFPQSLKGDWRPVPKGYNYCPTSAD
KNLHGDLIDIGLRLRAPKSFKGISADGWMCHAARWITTCDFRWYGPKYIT
HSIHSFRPSNDQCKEAIRLTNEGNWINPGFPPQSCGYASVTDSESVVVTV
TKHQVLVDEYSGSWIDSQFPGGSCTSPICDTVHNSTLWHADHTLDSICDQ
EFVAMDAVLFTESGKFEEFGKPNSGIRSNYFPYESLKDVCQMDFCKRKGF
KLPSGVWFEIEDAEKSHKAQVELKIKRCPHGAVISAPNQNAADINLIMDV
ERILDYSLCQATWSKIQNKEALTPIDISYLGPKNPGPGPAFTIINGTLHY
FNTRYIRVDIAGPVTKEITGFVSGTSTSRVLWDQWVPYGENSIGPNGLRK
TASGYKYPLFMVGTGVLDADIHKLGEATVIEHPHAKEAQKVVDDSEVIFF
GDTGVSKNPVEVVEGWFSGWRSSLMSIFGIILLIVCLVLIVRILIALKYC
CVRHKKRTIYKEDLEMGRFLGGLNYL.

VIRAL HEMORAGIC SEPTICEMIA FR-L59X GenBank: AAT01193.1:

(SEQ ID NO: 23)
MEWNTFFLVILIIIIKSTTSQITQRPPVENISTYHADWDTPLYTHPSNCR
EDSFVPIRPAQLRCPHEFEDINKGLVSVPTQIIHLPLSVTSVSAVASGHY
LHRVTYRVTCSTSFFGGQTIEKTILEAKLSRQEAVNEASKDHEYPFFPEP
SCIWMKNNVHKDITHYYKTPKTVSVDLYSRKFLNPDFIEGVCTTSPCQTH
WQGVYWVGATPTAHCPTSETLEGHLFTRTHDHRVVKAIVAGHHPWGLTMA
CTVTFCGTDWIKTDLGDLIKVIGQGGEKKLTPKKCVNTDIQMRGATDDFS
YLNHLITNMAPRTECLDAHSDITASGKISSFLLSKFRPSHPGPGKAHYLL
DGQIMRGDCDYEAVVSINYNSAQYKTVNNTWKSWKRIGNNTDGYDGMIFG
DKLVIPDIEKYQSIYDSGMLVQRNLVEVPHLSIVFVSNTSDLSTNHIHTN
LIPSDWSFHWSLWPSLSGMGIVGGAFLLLVLCCCCKASPPIPNYGIPMQQ
FSRNQMV.

DROSOPHILA MELANOGASTER SIGMA VIRUS HAP23 GenBank: ACU65437.1:

(SEQ ID NO: 24)
MVHYETHILFIHLWMLALIFITTSVWLAASQKTFTPDLVFPEMNRNSSWS
VANYGEILCPTSFQSYDPKKHQILTRVLVERPSLNTDTKVEGYTCHKVKY
ETICDMPWYFSPTISHSISPLRVKESECKDAIAEHQLGTHVSLSFPPEDC
SWNSVNTKAYEDIIVKDHPVMLDPYTNNYVDAIFPGGISSPGMGGTIHDD
MMWVSKDLAVSPECSGWQQSMGLIYSSRLYGEREPMLEVGSIHIEGHRDK
NLTLACRISFCGEIGVRFHDGEWMKVSVNLDHPNSVTFQVTDFPPCPPGT
TIQTAVVENINPEIQELTVNMMYRLKCQETISKMVSGLPTSALDLSYLIQ
VQEGPGIVYKREKGVLYQSVGMYQYIDTVTLNTEENQLGENARGQKVFWT
EWSDSPTRPDLQEGINGIVKYEGQIRVPLGMSLRLEAATELMWGHPVHTV
SHPILHVISNHTEQSVTTWNRGVNSTNLIGLATRSISGFYNDLKLYLILA
LIVVSIVALVVLDVIPFKYILFILCPPLLLCRFIKCSRRRPETGDRYHVE
YNRPGQVSSAF.

ORCHID FLECK VIRUS NCBI Reference Sequence: YP_{—}001294928.1:

(SEQ ID NO: 25)
MSTFCSLTVLMFLLISGFEALSLVPKTVCEKEVGLHVDEWLHSCLGACKS
SDNDLDMTPHMLMEPTLEYFKAFGYFIHVSTLTKSSHTFLLGGCHITSQE
TPSDSQELPTNMAENILRHGGPEGEIFMTKEPQCSLWSDNYVKGMLVKYH
RVILTVSHTKSGITVLYEQEGVIGHGKVGKTTSSSGTLVWDVRAQYPKCN
YRPTGVLSCKREESYIRCRGMSEEKISSITEDCGVIILTTDTRNIYGYHK
HDGALTTTQTDDNQIGMVKKIIEIENLMCHHLCESSSEEGGVTHHEYLVS
TPIGPWLAVSTESHRTMFMCTDESAVSLVVPVVLCGNGPLVKVRVNEREQ
WWNISSPYVGMGTHCVPGLSTGLSTVDGVINTWLGQVTIVNGSFTFKRDF
INKNFHGAFRPSQFSWSHKTGNDLEHIIDALNTQEKVLSHSHVIESHSVG
AGENAVHSFIGMFTTVFEWIQSLIPNVKGWIIKIFLWALLAALVILLLWI
LWKVLWLFIKSVFLRRTIQAVPTSENSDTSLNRAIHNWAKMD.

SINIPERCA CHUATSI RHABDOVIRUS NCBI Reference Sequence: YP_{—}802941.1:

(SEQ ID NO: 26)
MKLIIAPTLISQAIGYPLFVPIRLQGWHDVKLDTLMCPSYASELNKEAAW
PQIGLRHLAATDHYEVKGTICHKTTWVKTCDFRWYGPRYITTKISYAPVT
GLECQQAIVKASKDELETPYMPEDNCNWATISDNEKTFITVQKSNIFMDP
YNMVYVSTVLKGGRCASTVCPLEMHGGIWIPSEAPRESCKLGSSITSHIN
PNNASRLVTEASYLVTEYRRQLPFLGACRMSMCGEVGMRFKSGEWYKIES
SDGRVLSFIASVPMCDGELTVSIHDSSATYHKLSQEILDLSAQIACISEL
RRAREKKAVSNYLLSFLTPNHGGFGTAYRVLNGQLQASKATYVRVKLGAL
STATNWGQLDDGSAYSSEDVTGEIVNGPLFNGNRMDNGTLRVVQNAILGQ
TLEDEDLYEHSAKEILHPHLTILSSNESDVLSAFRPVGAQGDIIHAVGEW
VGTGVGGFIHTIVYLVILCGIIFLLYRCLPYFLKKRKSQSTSQMTPQMIP
LQQYQFVP.

CALIGUS ROGERCRESSEYI CROG-EVP-513-004 GenBank: ACO10239.1:

(SEQ ID NO: 27)
MKSVFFFFVILFNTMDADLSIPLGNTNNLPGLTTPLEPTVEGRSRTSRKE
KDFGEMTVLPLRRLEPWRECAIADLQCPPRYDFGEKIGSLITTEKLWPVR
GLSVLQEGYFCTKTSRDRTCSTSFFGSEDLSGSEEYLYPNDSDCLKEVKS
LESGRYSPPVWPEHTCAWMATRTVTLVQYQLNLHNVLWEDIGGTYHDAKL
KGGKCSTRICSTNNPGVLWIRGKKANHLLRPEDRLPCKIYENKTSSILQV
HCDYHHPLHFKVGACKFSYRGESGIRSEEGVGLAWDLKKGSKIARYVGPE
CDKKKTPIYQWSANSKFRYDAATKADDDLHARCLDALVRIREHKDISQWD
LGYFYPSSPGPFPAYRLNKSSKVLECSKYLFTLKEVKSGRSLVDYLPEES
IIPDPRTGEKLGVSGLIVKNDSSVYVPHQRSNQMISWEHHLGPKDKVEIS
RMSLTPNVDDNTLLFSEDPQNHATENGTSQATFLSHLGFGIEHLFYTLLG
LSIAGLVGVCVVKVCLENAFKCGCKYCSRSDLDDYSH.

TROUT RHABDOVIRUS903/87 GenBank: AAL35757.1:

(SEQ ID NO: 28)
MDLLLKILLIIIIIKEARTRIAFVPLDLGTWRATSVDQLDCPMQGDLVNQ
GTDAIEVRYQTAAWGLKNSVAGKLCVTAKWSITCDYRWYGSKYISTQIEY
LPTTPEMCKEAKRQLDRGELLSPHFSSENCGWNNVLTESETYTTIVSHPV
KLDAYSLTLIDNIFEGGKCHEEECPSLLHQGFWIADKNSLGLCKQLDSHI
GRLFKTGLRNSMGTVVRQQWDINSIFQPEIGREKNFNGACTMEFCGHQGL
RFADREWFKIPEQANQSLKKVFKDLPSCGTDNLVHSHDINTDVKEITEHV
HEVALHAICLQEVRRAKDSNTVSDWLLSMMTPFSEGAGPVYRIHNGKLES
TVGFYRKVLIEGGGTTEQLGIGFDKKPIKWDQFVVKTNDTRVQSMYNGNT
WDGEIHWAKNVLGSHILDEIAALEFDIPLIQHPHLDGILINETHVISSHH
PNGQGVNFVESVTHWAGGIWESVGSSAIIIAVILVVGFVLVKFCMRFSMP
VRRERREEGMMMLQRA.

HIRAME RHABDOVIRUS KOREA/CA 9703/1997 NCBI Reference Sequence: NP_{—}919033.1:

(SEQ ID NO: 29)
MDPRIMYYTVLLTTAARVYGQTIKPGVDSVSDQPTWANPLFTYPVDCPAA
KLSKVSPSQLRCPRIFDDENRGLVAYPAVIRSLSVGNNLGDIHTQGEYVH
KVLYRTTCSTGFFGGQTIEKALVEMKLAPREVGVYDTTTASALYFPAPRC
QWYTDNVHNDLTFYYTTAKSVLRDPYTLGFLDSDFIEGKCSKSPCQTHWS
NVVWKGDSGVAACDTGSEIKGHIFVDKTSHHVVKATSYGHHPWGLHRACM
ITFCGKPWIRTDLGDLIAIEYNGGATLLAFPACKDTTVGMRGSLDDFAYL
DDLVKSSESREECLEAHAEIIATNSVTPYLLSKFRSPHPGINDVYAMHDG
SIYHGKCMTVAIDEVSKDRRTYRAHQTSAFVAWGHPFGDEWGGFHGLHGN
DTPVIPDLEKYVAQYKVSMMDKMDIRPVPHPSVQILYNDTDTADITIRKI
DSFDLQSLNWSFWPSLSALGGVPILLALVFFLYCCMNRRPSMPAAPQEIP
MYHLASRG.

PIRY VIRUS UniProtKB/Swiss-Prot: Q85213.1:

(SEQ ID NO: 30)
MDLFPILVVVLMTDTVLGKFQIVFPDQNELEWRPVVGDSRHCPQSSEMQF
DGSRSQTILTGKAPVGITPSKSDGFICHAAKWVTTCDFRWYGPKYITHSI
HHLRPTTSDCETALQRYKDGSLINLGFPPESCGYATVTDSEAMLVQVTPH
HVGVDDYRGHWIDPLFPGGECSTNFCDTVHNSSVWIPKSQKTDICAQSFK
NIKMTASYPSEGALVSDRFAFHSAYHPNMPGSTVCIMDFCEQKGLRFTNG
EWMGLNVEQSIREKKISAIFPNCVAGTEIRATLESEGARTLTWETQRMLD
YSLCQNTWDKVSRKEPLSPLDLSYLSPRAPGKGMAYTVINGTLHSAHAKY
IRTWIDYGEMKEIKGGRGEYSKAPELLWSQWFDFGPFKIGPNGLLHTGKT
FKFPLYLIGAGIIDEDLHELDEAAPIDHPQMPDAKSVLPEDEEIFFGDTG
VSKNPIELIQGWFSNWRESVMAIVGIVLLIVVTFLAIKTVRVLNCLWRPR
KKRIVRQEVDVESRLNHFEMRGFPEYVKR.

VESICULAR STOMATITIS ALAGOAS VIRUS GenBank: ACB47442.1:

(SEQ ID NO: 2)
MTPAFILCMLLAGSSWAKFTIVFPQSQKGDWKDVPPNYRYCPSSADQNWH
GDLLGVNIRAKMPKVHKAIKADGWMCHAAKWVTTCDYRWYGPQYITHSIH
SFIPTKAQCEESIKQTKEGVWINPGFPPKNCGYASVSDAESIIVQATAHS
VMIDEYSGDWLDSQFPTGRCTGSTCETIHNSTLWYADYQVTGLCDSALVS
TEVTFYSEDGLMTSIGRQNTGYRSNYFPYEKGAAACRMKYCTHEGIRLPS
GVWFEMVDKELLESVQMPECPAGLTISAPTQTSVDVSLILDVERMLDYSL
CQETWSKVHSGLPISPVDLGYIAPKNPGAGPAFTIVNGTLKYFDTRYLRI
DIEGPVLKKMTGKVSGTPTKRELWTEWFPYDDVEIGPNGVLKTPEGYKFP
LYMIGHGLLDSDLQKTSQAEVFHHPQIAEAVQKLPDDETLFFGDTGISKN
PVEVIEGWFSNWRSSVMAIVFAILLLVITVLMVRLCVAFRHFCCQKRHKI
YNDLEMNQLRR.

PIKE FRY RHABDOVIRUS GenBank: ACP28001.1:

(SEQ ID NO: 31)
MSIYYALFMVSLMSGCESIPIFVPQNQDISWQPVIRPFNYQCPIHGNLPD
TFGLKSTKLTFWSPSVFSPGTVSGWICHAAEWKTTCDYRWYGPQYITHSI
HSIRPTIEECKRSIKQLESGTDEDLGFPPQSCGWASVTTVSNKNYKVVVH
PVHLEPYQGKWVDHEFLGGECNAPVCEMRGNHSIWLTDHILKEECDQHIE
ETTGTMYGNARRGDNLYVNNFIIDDHHRVYKFQDACRMKFCGMDGIKFLR
GDWIEKKGELARLHENVPDCADGTLISGHKPGADLVDTVFNLENLLEFTL
CESTKAKINRREEITSVDLSYLAPRVGGLGSIFRVRNGTLERGSTIYMRI
EVEGPIVPRLEGFDPRTNQSRVFWDDWDLDGEVYQGFNGIYKGQDQMIHI
PLNMIESGLVDEELQRSFQADTIPHPHFSDDGVSDEDIFFDNTGENGNPV
DAVVEWVSGWGTSLKFFGMTLIALVFLFIIIRVVIAVIYCLKKPKKTTVE
SREMRSLV.

Thus, the invention encompasses compositions and methods involving the use of Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, Pike Fry rhabdovirus, Wongabel virus, viral Hemoragic septicemia virus FR-L59X, and Flanders virus G proteins for pseudotyping retroviral, particularly lentiviral, vectors. Preferably, these proteins comprise or consist of the amino acid sequence of any of amino acid sequences 2 and 4-31.

The invention provides expression vectors expressing viral G proteins and lentivectors comprising viral G proteins. The lentivectors can be used in combination with lentivectors comprising viral G proteins. The invention especially provides methods for generating lentivectors and methods and uses of the lentiviral vectors in iterative administration, either for prevention or for treatment of a disease in a host, particularly in a mammalian host, and especially in human beings. A particular application of these vectors is to elicit an immune response to prevent or to treat a pathogenic state, including virus infections, parasite and bacterial infections or cancers, and preferably to elicit a protective, long-lasting immune response. According to a particular embodiment of the present invention, the designed vectors are especially of interest in the field of treatment or prevention against Immunodeficiency Virus and particularly against AIDS.

### Vectors Expressing Viral G Proteins

The invention encompasses expression vectors comprising a nucleotide sequence encoding a viral G protein. The viral G protein can be expressed from a polynucleotide comprising the coding sequence for the protein. Thus, the invention encompasses expression vectors that express the viral G protein.

Preferably, the viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, Pike Fry rhabdovirus, Wongabel virus, viral Hemoragic septicemia virus FR-L59X, and Flanders virus G proteins. Preferably, the viral G protein comprises or consists of the amino acid sequence of any of SEQ ID NOs 5-31.

More preferably, the viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, and Pike Fry rhabdovirus.

Preferably, the vectors can produce a titer of pseudotyped lentiviral vector particles of at least 5×10⁵, 10⁶, 2×10⁶, 5×10⁶, 8×10⁶, 10⁷, or 2×10⁷ TU/ml when co-transfected with a packaging vector and a lentivector. Within the context of this invention, whether a viral G protein expression vector “can produce a titer of pseudotyped lentiviral vector particles of at least 5×10⁵, 10⁶, 2×10⁶, 5×10⁶, 8×10⁶, 10⁷, or 2×10⁷ TU/ml when co-transfected with a packaging vector and a lentivector can be determined using the following assay procedure:

The lentiviral vectors are produced by transient transfection of HEK 293T cells using a standard calcium phosphate precipitation protocol. HEK 293T cells a seeded at 7×10⁶ cells in 10 cm² Tissue Culture Dish (BD Falcon) in 10 mL of Dubelcco's modified Eagle's medium supplemented with 10% fetal bovine serum, 1% L-Glutamine, 1% Penicillin-Streptomycin, and 1% Sodium Pyruvate and maintained 24 h in an incubator with humidified atmosphere of 5% CO₂ at 37° C. to adhere. For each vector produced, three tissue culture dishes are transfected as follows: the lentiviral backbone plasmid pFlap-ΔU3-CMV-GFP (10 μg in 10 μL), the pThV-ENV encoding envelope plasmid (5 μg in 5 μL), and the pThV-GP packaging plasmid (10 μg in 10 μL) are mixed with 353 μL of sterile distilled water and 125 μL of 1M CaCl₂. The DNA mix is then added drop to drop to 500 μL of 37° C. prewarmed HBS 2×pH=7.3 and the 1 mL of precipitate obtained is added to the culture medium of the cells containing 10% of fetal bovine serum (DMEM+Peni-Strepto 100 U final, L-Glutamine 2 mM final and Sodium pyruvate 1 mM final). The transfected cells are then incubated at 37° C. in 5% CO₂. The medium is replaced 24 h after transfection with 7 mL of harvest medium (DMEM: L-Glutamine 2 mM final and Sodium pyruvate 1 mM final) without serum and the viral supernatant is harvested after an additional 24 h, clarified by centrifugation 5 min. at 2500 rpm, and stored at −20° C. until assay.

For the quantification of infective particles, HEK 293T cells are seeded in 24-well plates at a density of 1×10⁵ cells per well in complete medium (DMEM+Peni-Strepto 100 U final, L-Glutamine 2 mM final and Sodium pyruvate 1 mM final) containing 10% FBS and incubated for 4 h to adhere. The cells are transduced by replacing the medium with 300 μl of dilutions 1/100, 1/300 and 1/900 of viral samples in complete medium, followed by incubation at 37° C., 5% CO₂ for 2 h. After adsorption, 1 mL of complete medium is added to each well. At 72 h posttransduction, the cells are trypsinized and resuspended in 300 μL of complete medium, and the percentage of cells expressing GFP was determined with a FACScalibur flow cytometer (BD Biosciences), using the FL1 channel. Two sets of three dilutions are performed for each sample tested. The values corresponding to a percentage of transduced cells less than 30% are used to calculate the approximate number of transducing units (TU) present in the viral suspension.

In one embodiment, the invention encompasses expression vectors comprising a nucleotide sequence encoding Alagoas VSV-G protein.

The Alagoas VSV-G protein can be expressed from a polynucleotide comprising the coding sequence for the protein. Thus, the invention encompasses expression vectors that express Alagoas VSV-G protein.

In one embodiment, the expression vectors encodes the amino acid sequence of SEQ ID NO:2. In one embodiment, the expression vectors contains the nucleotide sequence of SEQ ID NO:1. The expression vector is preferably a mammalian expression vector.

In various embodiments, the expression vector that express Alagoas VSV-G protein produces a bulk titer of at least 10⁶, 2×10⁶, 5×10⁶ or 10⁷ TU/ml of pseudotyped lentivector. The pseudotyped lentivector can be generated following the techniques illustrated in the examples herein.

The invention includes an expression vector expressing a VSV-G protein. The VSV-G glycoprotein can be from among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

A polynucleotide encoding a viral G protein can be inserted in a plasmid (viral G protein expression plasmid or pseudotyping plasmid) used for the preparation of a lentiviral vector. The polynucleotide encoding the viral G protein is under the control of regulatory sequences for the transcription and/or expression of the coding sequence, including optionally a polynucleotide such as a WPRE or Kozak sequence.

The invention encompasses an isolated polynucleotide which comprises a promoter suitable for the use in mammalian, especially in human cells, in vivo and the nucleic acid encoding Alagoas VSV-G protein or other viral G protein under the control of the promoter. The invention also concerns a plasmid containing this polynucleotide. Promoters can in particular be selected for their properties as constitutive promoters, tissue-specific promoters, or inducible promoters. The promoter is preferably a viral promoter, such as the strong cytomegalovirus (CMV) promoter. Preferably, the expression vector contains a poyadenylation signal downstream of the gene encoding the viral G protein. In some embodiments, the promoter is an RSV, Ubiquitin or EF1-α promoter.

The nucleotide sequence used for the expression of the viral G protein for pseudotyping the lentiviral vector particles is preferably modified with respect to the native nucleic acid encoding the viral G protein. The modification can be carried out to improve the codons usage (codon optimization) in the cells for the preparation of the vector particles.

Modification of the viral G protein can affect and especially improve its level of production in a cell host or their ability to pseudotype the vector particles possibly by improving the density of the viral G protein associated with the pseudotyped lentiviral vector particles. The modification can derive from a mutation in the amino acid sequence of the protein(s), for instance by addition, deletion or substitution of one or several nucleotides or nucleotidic fragments. Preferably, the modified VSV-G protein has 10, 9, 8, 7, 6, 5, 4, 3, 2, or 1 modified amino acids compared to the amino acid sequence of any of SEQ ID NOs 2 or 4-31.

### Cells Comprising Vectors

The invention further encompasses host cells comprising the vectors of the invention. In one embodiment, the vector is an expression vector comprising a nucleotide sequence comprising a codon optimized Alagoas vesicular stomatitis virus G protein gene. Preferably, the expression vector comprises a nucleotide sequence comprising the sequence of SEQ ID NO:1.

The cells can be generated by transfection or transduction of a cell with an expression vector comprising a nucleotide sequence encoding any of the viral G proteins referenced herein. The cells can transiently or stably express the viral G protein, either constitutively or inducibly. Preferably, the cells express an amino acid sequence selected from SEQ ID NOs 2 and 4-31.

Stable cell lines can be generated by routine techniques, by transduction or transfection. Cell lines containing the expression vector can be selected using a selectable marker.

Preferably, the expression vector is inducible. In various embodiments, the invention encompasses an inducible system utilizing a promoter requiring a protein not found in the host cell, for example, a T7 promoter. The inducible promoter functions in the presence of the protein, when provided to the cell. The protein (e.g., T7 polymerase) can be provided to the cell by transfection or transduction of the cells with a vector expressing the protein.

In various embodiments, the invention encompasses an inducible system utilizing Tet-On Systems. Tet-On Systems are inducible gene expression systems for mammalian cells. Target cells that express the a specific transactivator protein and contain a transgene under the control of a promoter (e.g., PTRE3G) will express high levels of the transgene, but only when cultured in the presence of doxycycline (Dox).

The transactivator protein is a transcriptional regulator that display high sensitivity to doxycycline (Zhou, X., Vink, M., Klave, B., Berkhout, B. & Das, A. T. (2006) Optimization of the Tet-On system for regulated gene expression through viral evolution. Gene Ther. 13(19):1382-1390).

The inducible promoter (PTRE3G) provides for very low basal expression and high maximal expression after induction (Rainer Loew, Niels Heinz 1, 3, Mathias Hampf4, Hermann Bujard2, Manfred Gossen4, 5. (2010) Improved Tet-responsive promoters with minimized background expression. BMC Biotechnology. 10:81). It consists of 7 repeats of a 19 bp tet operator sequence located upstream of a minimal CMV promoter. In the presence of Dox, the transactivator binds specifically to PTRE3G and activates transcription of the downstream transgene. PTRE3G lacks binding sites for endogenous mammalian transcription factors, so it is virtually silent in the absence of induction.

### Lentivectors Comprising Viral G Proteins

The invention encompasses lentivectors comprising a viral G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, Pike Fry rhabdovirus, Wongabel virus, viral Hemoragic septicemia virus FR-L59X, and Flanders virus G proteins. Preferably the G protein comprises or consists of the amino acid sequence of any of SEQ ID NOs 2 or 4-31. Preferably, the lentivector comprises an Alagoas VSV-G protein.

The lentivector can be integrative or non-integrative. The lentiviral vectors are pseudotyped lentiviral vectors (i.e. “lentiviral vector particles”) bearing G envelope proteins from other viruses.

Preferably, the sequences of the original lentivirus encoding the lentiviral proteins are essentially deleted from the genome of the vector or, when present, are modified, and particularly prevent expression of biologically active Pol antigen and optionally of further structural and/or accessory and/or regulatory proteins of the lentivirus.

Within the context of this invention, a “lentiviral vector” means a non-replicating vector for the transduction of a host cell with a transgene comprising cis-acting lentiviral RNA or DNA sequences, and requiring lentiviral proteins (e.g., Gag, Pol, and/or Env) that are provided in trans. The lentiviral vector contains cis-acting packaging sequences, but lacks expression of functional Gag, Pol, and Env proteins. The lentiviral vector may be present in the form of an RNA or DNA molecule, depending on the stage of production or development of the vector.

The lentiviral vector can be in the form of a recombinant DNA molecule, such as a plasmid. The lentiviral vector can be in the form of a lentiviral particle vector, such as an RNA molecule(s) within a complex of lentiviral and other proteins. Typically, lentiviral particle vectors, which correspond to modified or recombinant lentivirus particles, comprise a genome which is composed of two copies of single-stranded RNA. These RNA sequences can be obtained by transcription from a double-stranded DNA sequence inserted into a host cell genome (proviral vector DNA) or can be obtained from the transient expression of plasmid DNA (plasmid vector DNA) in a transformed host cell.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene.

The “vector genome” of the vector particles also comprises a polynucleotide or transgene of interest. In a particular embodiment, the transgene is also devoid of a polynucleotide encoding biologically active POL proteins. A biologically active POL antigen comprises the viral enzymes protease (RT), reverse tanscriptase (RT and RNase H) and integrase (IN) produced by cleavage of the GAG-POL polyprotein. The POL antigen is not biologically active, when the biological activity of at least one of these enzymes is not enabled. The biological activity is described with these enzymes in Fields (Virology—Vol 2 Chapter 60, pages 1889-1893 Edition 1996). In a particular embodiment, the polynucleotide or transgene in the vector genome is devoid of the functional pol gene, and especially does not contain a complete pol gene.

The vector genome as defined herein contains, apart from the so-called heterologous polynucleotide of therapeutic interest placed under control of regulatory sequences, the sequences of the lentiviral genome which are non-coding regions, and are necessary to provide recognition signals for DNA or RNA synthesis and processing. These sequences are cis-acting sequences. The structure and composition of the vector genome used to prepare the lentiviral vectors of the invention are based on the principles described in the art. Examples of such lentiviral vectors are disclosed in (Zennou et al, 2000; Firat H. et al, 2002; VandenDriessche T. et al). Especially, minimum lentiviral gene delivery vectors can be prepared from a vector genome, which only contain, apart from the heterologous polynucleotide of therapeutic interest under control of regulatory sequences, the sequences of the lentiviral genome which are non-coding regions of the genome, necessary to provide recognition signals for DNA or RNA synthesis and processing. Hence, a vector genome can be a replacement vector in which all the viral protein coding sequences between the 2 long terminal repeats (LTRs) have been replaced by the polynucleotide of interest.

The polynucleotide encoded (contained) by the lentiviral vector particles is “heterologous” because it is brought as an insert in the vector genome construct. In particular embodiments, the genome vector and the polynucleotide can originate from the same group of lentiviruses, even from the same type.

In a particular embodiment of the invention, the heterologous determined polynucleotide, encodes one or several polypeptides comprising at least one antigen derived from a GAG antigen of an Immunodeficiency Virus. Especially, the antigen is or comprises one or more immunogenic epitopes. When intended for the design of a vector suitable for a human host, the GAG antigen can be derived from a GAG polyprotein of a Human Immunodeficiency Virus, especially HIV-1 or HIV-2. In a particular embodiment, the encoded antigen derived from Gag, especially immunogenic epitope(s) derived from Gag, is a biologically non-functional Gag.

In a particular embodiment, the vector genome is defective for the expression of biologically functional Gag, and advantageously for biologically functional PoI and Env proteins. The 5′ LTR and 3′ LTR sequences of the lentivirus can be used in the vector genome. Preferably, the 3′-LTR is modified with respect to the 3′LTR of the original lentivirus, particularly in the U3 region. The 5′LTR can also be modified, particularly in its promoter region.

In a preferred embodiment, the 3′ LTR sequence of the lentiviral vector genome is devoid of at least the activator (enhancer), and preferably also the promoter of the U3 region. In another particular embodiment, the 3′ LTR region is devoid of the U3 region (delta U3). In this respect, reference is made to WO 01/27300 and WO 01/27304.

In a particular embodiment, in the vector genome, the U3 region of the LTR 5′ is replaced by a non lentiviral U3 or by a promoter suitable to drive tat-independent primary transcription. In such a case, the vector is independent of tat transactivator.

In a particular embodiment the vector genome is devoid of the coding sequences for Vif-, Vpr, Vpu- and Nef-accessory genes (for HIV-1 lentiviral vectors), or of their complete or functional genes.

In a preferred embodiment, the vector genome of the lentiviral vector particles comprises, as an inserted cis-acting fragment, at least one polynucleotide consisting of or comprising the DNA flap. In a particular embodiment, the DNA flap is inserted upstream of the polynucleotide of interest. Preferably, the DNA flap is located in an approximate central position in the vector genome. A DNA flap suitable for the invention can be obtained from a retrovirus, especially from a lentivirus, in particular a human lentivirus, or from a retrovirus-like organism such as retrotransposon. It can be alternatively obtained from the CAEV (Caprine Arthritis Encephalitis Virus) virus, the EIAV (Equine Infectious Anaemia Virus) virus, the Visna virus, the SIV (Simian Immunodeficiency Virus) virus or the FIV (Feline Immunodeficiency Virus) virus. The DNA flap can be prepared synthetically (chemical synthesis) or by amplification of the DNA, such as by polymerase chain reaction (PCR). In a more preferred embodiment, the DNA flap is obtained from an HIV retrovirus, for example HIV-1 or HIV-2 virus including any isolate of these two types.

The DNA flap (defined in Zennou V. et al., 2000, Cell vol 101, 173-185 or in WO 99/55892 and WO 01/27304, which are hereby incorporated by reference), is a structure which is central in the genome of some lentiviruses especially in HIV, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral-derived vectors, the polynucleotide enabling the DNA flap to be produced during reverse-transcription, stimulates gene transfer efficiency and complements the level of nuclear import to wild-type levels (Zennou et al., Cell, 2000).

Sequences of DNA flaps are well-known in the art, for example, in the above cited patent applications. They are preferably inserted as fragment comprising the DNA Flap into the vector genome in a position which is preferentially near the center of the vector genome. Alternatively, they can be inserted immediately upstream from the promoter controlling the expression of the polynucleotide of the invention. The fragments comprising the DNA flap, inserted in the vector genome can have a sequence of about 80 to about 200 bp, depending on its origin and preparation. According to a particular embodiment, a DNA flap has a nucleotide sequence of about 90 to about 140 nucleotides.

In HIV-1, the DNA flap is a stable 99-nucleotide-long plus strand overlap. When used in the genome vector of the lentiviral vector of the invention, it can be inserted as a longer sequence, especially when it is prepared as a PCR fragment. A particular appropriate polynucleotide comprising the structure providing the DNA flap is a 178-base pair polymerase chain reaction (PCR) fragment encompassing the cPPT and CTS regions of the HIV-1 DNA (Zennou et al 2000).

This PCR fragment can especially be derived from infective DNA clone of HIV-1 LAI, especially pLAI3 of HIV1, as a fragment corresponding to the sequence from nucleotide 4793 to 4971. If appropriate, restriction sites are added to one or both extremities of the obtained fragment, for cloning. For example, Nar I restriction sites can be added to the 5′ extremities of primers used to perform the PCR reaction.

The DNA flap used in the genome vector and the Gag and Pol polyproteins of the lentiviral vector particles should originate from the same lentivirus sub-family or from the same retrovirus-like organism. Preferably, the other cis-activating sequences of the genome vector also originate from the same lentivirus or retrovirus-like organism, as the one providing the DNA flap.

The vector genome can further comprise one or several unique restriction site(s) for cloning the polynucleotide of interest.

Preferably, the pseudotyped lentiviral vector is a replication-incompetent lentiviral vector as a result of the fact that gag and pol functional genes are exclusively provided in trans and therefore not present on the vector genome. In such a case, when the lentiviral vector has been administered to the host, it is not capable of replicating in the host cells. Accordingly, it provides the polynucleotide of therapeutic interest into the host cells for expression but does not form further lentiviral vector particles (“replication-incompetent”). The lentivector can be integrative or non-integrative.

Preferably, the vector genome comprises a psi (ψ) packaging signal. The packaging signal is derived from the N-terminal fragment of the gag ORF. In a particular embodiment, its sequence could be modified by frameshift mutation(s) in order to prevent any interference of a possible transcription/translation of gag peptide, with that of the transgene.

The vector genome can optionally also comprise elements selected from a splice donor site (SD), a splice acceptor site (SA) and/or a Rev-responsive element (RRE).

According to a particular embodiment, the vector plasmid (or added genome vector) comprises the following cis-acting sequences for a transgenic expression cassette: an LTR sequence (Long-Terminal Repeat), preferably deleted in the U3 region; a ψ region; and RRE sequence; and a DNA flap sequence (cPPT/CTS). Optionally, the WPRE cis-active sequence (Woodchuck hepatitis B virus Post transcriptional-Response Element) also added to optimize stability of mRNA (Zufferey et al., 1999).

Preferably, the lentivector comprises a heterologous polynucleotide. The heterologous polynucleotide can encode at least one antigenic polypeptide. The lentiviral vector genome can comprise less than a complete lentiviral gag, pol or env coding polynucleotide, meaning that the lentiviral vector genome comprises a polynucleotide shorter than the lentiviral gag, pol or env genes. Therefore, the gag coding sequence is shorter than 1500 for HIV-1 or HIV-2; the pol coding sequence is shorter than 3000 for HIV-1 and 3300 for HIV-2; the env coding sequence is shorter than 2700 for HIV-1 and 2500 for HIV-2. This size refers to the longest continuous nucleotide sequence found as such in the native lentiviral genome. However, in another particular embodiment, the lentiviral genome is devoid of all endogenous coding lentiviral sequences.

According to another particular aspect of the invention, the heterologous polynucleotide encodes a polypeptide (“heterologous polypeptide”) that is a tumor associated antigen (TAA) or a fragment thereof. Non-limiting known examples of TAA are especially: mutated peptides found in melanoma such as βcatetin, MART-2, or leukemia such as brc-abl, tissue specific proteins such as gp100, MART-1, tyrosinase, found in melanoma, or PSA, PAP, PSM, PSMA found in prostate cancer, cancer-testis antigen such as MAGE, molecules related to tumorigenesis such as Survivin, hTERT, found in various cancers, mucins like MUC-1 found in breast, ovarian or pancreas cancer, viral proteins of virus that transforms a normal cell in tumor cell (tumor virus) including those of HPV (Human Papilloma Virus), especially HPV16 or HPV18, including the HPV16-E7 antigen (found expressed in cervical cancer), EBV (Epstein-Barr virus) causing lymphoma including EBV-EBMA protein (in lymphoma), HBV (Hepatitis B Virus), HCV (Hepatitis C Virus), HHV (Human Herpes Virus) such as HHV8 or HTLV (Human T Leukemia Virus) such as HTLV-1, such HTLV-1 tax protein (in Acute T Leukemia). In a particular embodiment, the polynucleotide of interest encodes human antigens.

The heterologous polynucleotide can encode at least one polypeptide that is an artificial (non-natural) polypeptide, preferably a multiepitope polypeptide. This multiepitope polypeptide can encode at least two epitopes, originating from a pathogenic organism, including viruses, and/or of tumoral-origin.

The heterologous polynucleotide can be inserted in the vector genome, under the control of regulatory sequences for transcription and expression, including a promoter and an enhancer. In a particular embodiment, the regulatory sequences are not of lentiviral origin. Suitable promoters encompass CMV, also referred to as CMVie promoter, or EF1α promoter, CGA promoter, CD11c promoter and house keeping gene promoters such as PGK promoter, ubiquitin promoter, actin promoter, histone promoter, alpha-tubulin promoter, beta-tubulin promoter, superoxide dismutase 1 (SOD-1) promoter, dihydrofolate reductase (DHFR) promoter, hypoxanthine phosphorybosyltransferase (HPRT) promoter, adenosine deaminase promoter, thymidylate synthetase promoter, dihydrofolate reductase P1 promoter, glucose-6-phosphate dehydrogenase promoter or nucleolin promoter. Other suitable promoters encompass the promoters of the following genes: PPI (preproinsulin), thiodextrin, HLA DR invariant chain (P33), HLA DR alpha chain, Ferritin L chain or Ferritin H chain, Beta 2 microglobulin, Chymosin beta 4, Chymosin beta 10, or Cystatin Ribosomal Protein L41.

The invention encompasses compositions comprising lentiviral vector particles pseudotyped with a viral. Preferably, the composition comprises at least 10⁵, 5×10⁵, 10⁶, 5×10⁶, 10⁷, 5×10⁷, 10⁸, 5×10⁸, 10⁹, 5×10⁹, or 10¹⁰ TU/ml of lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, Pike Fry rhabdovirus, Wongabel virus, viral Hemoragic septicemia virus FR-L59X, and Flanders virus G proteins.

More preferably, the G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G proteins. Preferably, the amino acid sequence is selected from any of SEQ ID NOs 5-31.

In one embodiment, the composition has not been concentrated (“unconcentrated composition”). An “unconcentrated composition” comprising lentiviral vector particles refers to a composition comprising lentiviral vector particles that, although they may be purified from cells, have not been through a concentration step. For example, a composition comprising a cell-free supernatant of producer cells is an “unconcentrated composition”. Preferably, the unconcentrated composition comprises at least 10⁵, 5×10⁵, 10⁶, 5×10⁶, 10⁷, 5×10⁷, 10⁸, 5×10⁸, 10⁹, 5×10⁹, or 10¹⁰ TU/ml of lentiviral vector particles pseudotyped with a viral G protein.

The invention also encompasses lentiviral vector particles pseudotyped with a viral G protein that have not been through a concentration step. These are referred to as “unconcentrated lentiviral vector particles.”

In one embodiment, the composition has been concentrated (“concentrated composition”) 10, 100, or 1000-fold. A “concentrated composition” comprising lentiviral vector particles refers to a composition comprising lentiviral vector particles that have been through at least one concentration step, such as by ultracentrifugation and resuspension in a smaller volume. Similarly, filters can be used to reduce volume and concentrate the lentiviral vector particles. The fold concentration refers to the reduction in volume from the original cell supernatant. Preferably, the concentrated composition comprises at least 10⁸, 5×10⁸, 10⁹, 5×10⁹, or 10¹⁰ TU/ml of lentiviral vector particles pseudotyped with a viral G protein.

The invention also encompasses lentiviral vector particles pseudotyped with a viral G protein that have been through a concentration step. These are referred to as “concentrated lentiviral vector particles.”

A composition of the invention can comprise at least 10⁵, 5×10⁵, 10⁶, 5×10⁶, 10⁷, or 2×10⁷ TU/ml of unconcentrated lentiviral vector particles. In other embodiments, a composition of the invention can comprise at least 10⁸, 5×10⁸, 10⁹, 5×10⁹, or 10¹⁰ TU/ml of concentrated lentiviral vector particles.

In some embodiments, the composition can be a concentrated (or unconcentrated) composition, but refer to the titer of the lentiviral vector particles in unconcentrated form. Preferably, the unconcentrated form of the lentiviral vector particles comprises at least 10⁵, 5×10⁵, 10⁶, 5×10⁶, or 10⁷ TU/ml of lentiviral vector particles pseudotyped with a viral G protein.

The lentivectors comprising VSV-G Alagoas or viral G proteins can be generated by techniques known in the art. For example, transient cotransfections or the use of packaging cell lines expressing VSV-G Alagoas proteins and/or Gag and Pol proteins can be used to generate the lentivectors. Preferably, the packaging cell line expresses an inducible viral G protein.

### Methods of Making Pseudotyped Lentivectors

The invention also encompasses methods for using an expression vector encoding a viral G protein to generate lentiviral vectors. In one embodiment, the invention encompasses co-expressing a lentivector, a packaging vector(s) encoding lentiviral Gag and Pol proteins, and an expression vector encoding a viral G protein together in a cell.

The lentivector comprises cis-acting sequences for packaging and reverse transcription, including a ψ site and primer binding site. Preferably, the lentiviral vector comprises two HIV-1 LTR sequences. In one embodiment, one of the LTRs is deleted for U3 and R sequences. Preferably, the lentiviral vector comprises a central polypurine tract (cPPT) and a central terminal sequence (CTS). The lentiviral vector preferably encodes a lentiviral or non-lentiviral protein, such as a selectable marker, vaccine antigen, or tumor antigen.

In one embodiment, the lentivector comprises one or more HIV antigen, preferably an HIV-1 antigen. Most preferably, the antigen is a Gag, Pol, Env, Vif, Vpr, Vpu, Nef, Tat, or Rev antigen. The antigen can be a single antigen, a mix of antigens, an antigenic polypeptide, or a mix of antigenic polypeptides from these proteins. In a preferred embodiment, the lentiviral vector comprises an HIV-1 p24 Gag antigen.

In one embodiment, the invention encompasses a lentiviral vector comprising an promoter that comprises an NF-Kb binding site, an interferon sensitive response element (ISRE), and an SXY module (SXY). Examples are the β2m promoter and the MHC class I gene promoters. These promoters are generally cloned or reproduced from the promoter region of a gene encoding a protein β2m or a MHC class I protein, or referred to as putatively encoding such proteins in genome databases (ex: NCBI polynucleotide database http://www.ncbi.nlm.nih.gov/guide/dna-rna). Both β2m and class I MHC proteins enter the Major Histocompatibility Complex (MHC). β2m and class I MHC promoter sequences are also usually referred to as such in genome databases—i.e. annotated as being β2m and class I MHC promoter sequences.

In one embodiment, the packaging vector(s) and the lentiviral vector are introduced together into a cell to allow the formation of lentiviral vector particles containing the Gag protein produced by the packaging vector and the nucleic acid produced by the lentiviral vector. Preferably, this is achieved by cotransfection of the cells with the packaging vector(s) and the lentiviral vector. The cells can also be transfected with a nucleic acid encoding a viral G protein. Preferably, the lentiviral vector particles are capable of entry, reverse transcription, and expression in an appropriate host cell.

In one embodiment, the expression vector encoding a viral G protein, the packaging vector(s), or the lentiviral vector is stably integrated into cells, and the non-integrated vectors are transfected into the cells to allow the formation of lentiviral vector particles. All of the different permutations of this embodiment are apparent to the skilled artisan and are specifically contemplated.

In one embodiment, the method further comprises collecting the lentiviral vector particles produced by the cells.

Preferably, the titer of the lentiviral vector particles produced by the cells is at least 10⁵, 5×10⁵, 10⁶, 2×10⁶, 5×10⁶, 8×10⁶, 10⁷, or 2×10⁷ TU/ml.

In one embodiment, the lentiviral vector particles are concentrated by ultracentrifugation. Preferably, the titer of the concentrated lentiviral vector particles is at least 10⁸, 5×10⁸, 10⁹, 5×10⁹, 10¹⁰ TU/ml.

Most preferably, the lentiviral vector particles produced by the cells have a titer of least 10⁵, 5×10⁵, 10⁶, 5×10⁶, 10⁷, 5×10⁷, 10⁸, 5×10⁸, 10⁹, 5×10⁹, or 10¹⁰ TU/ml of lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins.

### Combinations of Lentivectors

The invention includes combinations of lentiviral vectors, which can provide an efficient prime-boost system for use for iterative administrations, enabling successively priming and boosting the immune response in a host, especially after injections in a host in need thereof. “Iterative” means that the active principle, i.e., the heterologous polynucleotide contained in the lentiviral vector of the invention is administered twice or more, such as three or four times, to the host, as a result of the administration of lentiviral vectors disclosed herein.

The invention is accordingly directed to a combination of compounds comprising at least: (i) lentiviral vector particles (also designated as lentiviral vectors or lentivectors) pseudotyped with a first viral G protein and (ii) lentiviral vector particles pseudotyped with a second viral G protein different from the first viral G protein, wherein the lentiviral vector particles of (i) and (ii) encode (i.e., contain) a heterologous polynucleotide which is in particular a recombinant polynucleotide (or transgene) encoding one or several polypeptides.

Preferably, the first and/or second viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA, Pike Fry rhabdovirus, Wongabel virus, viral Hemoragic septicemia virus FR-L59X, and Flanders virus G proteins.

In one embodiment, the first viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins and the second viral G protein is selected from Indiana, New Jersey, Cocal, SVCV, or Isfahan VSV-G proteins.

In one embodiment, the second viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins and the first viral G protein is selected from Isfahan, Ind., New Jersey, Cocal, or SVCV-G proteins.

Preferably, the lentiviral vector particles have a titer of least 10⁵, 5×10⁵, 10⁶, 5×10⁶, 10⁷, 5×10⁷, 10⁸, 5×10⁸, 10⁹, 5×10⁹, or 10¹⁰ TU/ml.

The invention is further directed to a combination of compounds comprising at least: (i) lentiviral vector particles (also designated as lentiviral vectors or lentivectors) pseudotyped with a first determined VSV-G protein; (ii) lentiviral vector particles pseudotyped with a second determined VSV-G protein different from the first determined VSV-G protein; wherein the lentiviral vector particles of (i) and (ii) encode (i.e., contain) a heterologous determined polynucleotide which is in particular a recombinant polynucleotide (or transgene) encoding one or several polypeptides and; wherein the first and second pseudotyping envelope protein(s) do not sero-neutralize with each other and are suitable for in vivo transduction of mammalian cells.

In preferred embodiments of the invention, the first or second determined VSV-G protein is VSV-G Alagoas protein.

The expression “combination of compounds” or “kit of compounds” means that the lentiviral vectors constituting active ingredients of the kits or combinations, are provided as separate compounds in the kit or combination, and are intended for separate administration to a host, especially separate administration in time. Accordingly the invention enables to perform a prime-boost administration in a host in need thereof, where the first administration step elicits an immune, especially cellular, immune response and the later administration step(s) boost(s) the immune reaction.

The compounds of the kit can be provided separately to the host in need thereof, especially to a mammalian host, in particular a human patient. The lentiviral vectors can be provided in separate packages or can be presented in a common package for a separate use thereof. The notice included in the packages and comprising the directions for use can indicate that the lentiviral vector particles which are pseudotyped with distinct VSV-G proteins are for separate administration in time, especially for priming and subsequently boosting an immune reaction in a host.

In preferred embodiments, the first and second VSV-G proteins, and if any the third or more VSV-G proteins, are selected for their capacity not to sero-neutralize with each other (i.e., not to cross-react). In these embodiments, each of the VSV-G proteins, used for pseudotyping the vector particles in the combination, does not react with and especially is not recognized by antibodies directed against the previously administered VSV-G protein(s). Accordingly, each of the first and second and if any the third or further, viral envelope protein(s), when administered within a lentiviral vector, does not elicit the production of antibodies, that recognize the subsequently administered VSV-G protein(s), where such production of the anti-VSV-G antibodies (so-called antivector immunity) would result in a failure to elicit an immune response against the product expressed from the polynucleotide.

In addition to Vesicular stomatitis Alagoas virus (VSAV), VSV strains include several serotypes that can provide envelope protein(s) for the preparation of the lentiviral vector. The VSV-G glycoprotein can especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

Most preferably, the G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins.

In one embodiment, when lentivector particles are successively administered which have different pseudotyping envelopes, a viral G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins is used in a first administration.

Preferably, the second administration of lentivector particles is pseudotyped with a second viral G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins that is different than the viral G protein used in the first administration.

Subsequent administrations of lentivector particles are preferably pseudotyped with a viral G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Pity virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins that is different than the viral G protein used in the first and second administrations.

In a preferred embodiment, Alagoas VSV-G proteins are used in a first administration. Preferably, the second administration of lentivector particles is pseudotyped with Indiana, New Jersey, Isfahan, Cocal, or SVCV VSV-G proteins. Subsequent administrations of lentivector particles are preferably pseudotyped with Indiana or New Jersey VSV-G proteins. Other orders of administration can be derived from FIG. 3 by avoiding those combinations that exhibit cross sero-neutralization.

In another embodiment, when lentivector particles are successively administered which have different pseudotyping envelopes, Alagoas VSV-G proteins are used in a second administration. Preferably, the first administration of lentivector particles is pseudotyped with Isfahan, Ind., New Jersey, Cocal, or SVCV G proteins. Subsequent administrations of lentivector particles are preferably pseudotyped with Isfahan, Ind. or New Jersey VSV-G proteins. Other orders of administration can be derived from FIG. 3 by avoiding those combinations that exhibit cross sero-neutralization.

### Administration of Lentivectors Comprising Viral G Proteins

Lentivectors comprising Alagoas VSV-G and other viral G proteins can be administered to a host by techniques known in the art.

Preferably, the viral G protein is selected from Yug Bogdanovac virus, VSIV-3 86 Agulhas NegrasB, VSIV-2 86 MaipuE, Vesicular stomatitis Alagoas virus, VSIV-2 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, Pike Fry rhabdovirus, Wongabel virus, viral Hemoragic septicemia virus FR-L59X, and Flanders virus G proteins. Preferably the viral G protein comprises or consists of the amino acid sequence of any of SEQ ID NOs 2, or 4-31.

The invention encompasses Alagoas VSV-G and other viral G proteins for iterative administrations with lentivectors to treat a host, including a human. The invention encompasses the use of Alagoas VSV-G and other viral G proteins for iterative administrations with lentivectors to treat a host, including a human. The invention further encompasses the use of Alagoas VSV-G and other viral G proteins in the preparation of a composition or vaccine for iterative administrations with lentivectors to treat a host, including a human.

The compositions of the invention can be injected in a host via different routes: subcutaneous (s.c.), intradermal (i.d.), intramuscular (i.m.) or intravenous (i.v.) injection, oral administration and mucosal administration, especially intranasal administration or inhalation. The quantity to be administered (dosage) depends on the subject to be treated, including considering the condition of the patient, the state of the individual's immune system, the route of administration and the size of the host.

Preferred dosage ranges for administration include a dose of at least 10⁶, 2×10⁶, 5×10⁶, 10⁷, 5×10⁷, 10⁸, 5×10⁸, 10⁹, 5×10⁹, or 10¹⁰ Transduction units (TU) of pseudotyped lentivector. TU can be determined by evaluating the in vitro potency of lentiviral particles. This quantification of the effective vectors is obtained after transduction of permissive cells, either by quantification of the integrated proviral DNA by qPCR (Taqman, Sybergreen) or by FACS analysis measuring the expression of a transgenic protein expressed by the vector.

According to preferred embodiments of the invention, additional administration steps are performed in order to boost the immune reaction further. The second administration can be of the same or a different dosage as the first administration.

The time between the two first administration steps can be in the range of 3 to 12 weeks or more depending on the response to the prime and on the indication. The time between the first boost and the last boosting step can be in the range of a few weeks, especially more than 12 weeks, for example 6 months, and even can be one or even several years.

### EXAMPLES

### Example 1

### Cell Lines

HEK 293T (human embryonic kidney cell line, ATCC CRL-11268, (Graham et al. 1977)) cells were maintained in Dubelcco's modified Eagle's medium (DMEM/High modified, Hyclone) supplemented with 10% fetal bovine serum (FBS, PAA), 1% L-Glutamine (Eurobio), 1% Penicillin-Streptomycin (Gibco by Life technologies) and 1% Sodium Pyruvate (Gibco by Life technologies).). The cell line was kept in an incubator with humidified atmosphere of 5% CO₂ at 37° C.

### Example 2

### Plasmids Construction

PCR amplification of the proviral region of the pTRIPΔU3-CMV-GFP (Naldini, L. et al. In vivo gene delivery and stable transduction of nondividing cells by a lentiviral vector. Science. 272, 263-267, 1996) was performed using direct (5′-CTTACTAGTTGGAAGGGCTAATTCACTCCCAAC-3′; SEQ ID NO:32) and reverse (5′-CATTCTAGAACTGCTAGAGATTTTCCACACTG-3′; SEQ ID NO:33) oligonucleotides encompassing respectively the Spel and Xbal restriction sites. The resulting fragment was digested and cloned between the Spel and Xbal sites of the pVAX-1 plasmid (Invitrogen, Lifetech) from which the Mlul site have been deleted. The resulting plasmid was named pFLAP-CMV-GFP. The SV40 sequence was amplified by PCR from the pTRIPΔU3-CMV-GFP plasmid (using the 5′-TACCCCGGGCCATGGCCTCCAAAAAAGCCTCCTCACTACTTC-3′; SEQ ID NO:34 and 5′-ACTCCCGGGTAATTTTTTTTATTTATGCAGAGGCCGAGGCCGCC-3′; SEQ ID NO:35 oligonucleotides), and cloned into the Pml1 site of the pFLAP-CMV-GFP, the resulting plasmid being then named pFLAP-CMV-GFP-SV. The CMV promoter was amplified with direct (5′-TACACGCGTGGAGTTCCGCGTTACATAACTTACGG-3′; SEQ ID NO:36) and reverse (5′-CGTGGATCCGATCGCGGTGTCTTCTATGGAGGTCAAAAC-3′; SEQ ID NO:37) oligonucleotides encompassing the Mlul and BamHI sites, respectively. The resulting fragment was cloned back between the Mlul and BamHI sites of the pFlap-CMV-GFP-SV allowing the easy replacement of the promoters inside the lentiviral vectors.

All the glycoproteins sequences (codon optimized for human used) were purchased by GeneArt (Lifetech) and cloned downstream the CMV promoter in the pVAX.1 plasmid (Lifetech), between the BamHI and EcoRI restriction sites.

### Example 3

### Lentiviral Production

### Bulks:

The lentiviral vectors were produced by transient transfection of HEK 293T cells using a standard calcium phosphate precipitation protocol. HEK 293T cells were seeded at 7×10⁶ cells in 10 cm² Tissue Culture Dish (BD Falcon) in 10 mL of complete culture medium and maintained 24 h in an incubator with humidified atmosphere of 5% CO₂ at 37° C. to adhere. For each vector produced, three tissue culture dishes were each transfected as following: the lentiviral backbone plasmid pFlap-ΔU3-CMV-GFP (10 μg), the pThV-ENV encoding envelope plasmid (5 μg), and the pThV-GP packaging plasmid (10 μg) were mixed with 353 μL of sterile distilled water (Gibco by Life Technologies) and 125 μL of CaCl₂ (Fluka). The DNA mix is then added drop to drop to 500 μL of 37° C. prewarmed HBS 2×pH=7.3 and the 1 mL of precipitate obtained was added to the culture medium of the cells. The transfected cells were then incubated at 37° C., 5% CO₂. The medium was replaced 24 h after transfection by 7 mL of harvest medium without serum and the viral supernatant was harvested after an additional 24 h, clarified by centrifugation 5 min. at 2500 rpm and stored at −20° C.

### Ultraconcentrated Vectors:

The lentiviral vectors were produced by transient transfection of HEK 293T cells using a standard calcium phosphate precipitation protocol. HEK 293T cells were seeded at 7×10⁶ cells in 10 cm² Tissue Culture Dish (BD Falcon) in 10 mL of complete culture medium and maintained 24 h in an incubator with humidified atmosphere of 5% CO₂ at 37° C. to adhere. For each vector produced, thirty tissue culture dishes were each transfected as following: the lentiviral backbone plasmid pFlap-ΔU3-CMV-GFP (10 μg), the pThV-ENV encoding envelope plasmid (5 μg), and the pThV-GP packaging plasmid (10 μg) were mixed with 353 μL of sterile distilled water (Gibco by Life Technologies) and 125 μL of CaCl₂ (Fluka). The DNA mix is then added drop to drop to 500 μL of 28° C. prewarmed HBS 2×pH=7.3 and the 1 mL of precipitate obtained was added to the culture medium of the cells. The transfected cells were then incubated at 37° C., 5% CO₂. The medium was replaced 24 h after transfection by 7 mL of harvest medium without serum and the viral supernatant was harvested after an additional 24 h, clarified by centrifugation 5 min. at 2500 rpm. The harvest clarified bulk (210 mL) is then treated 30 min with DNase (Roche) in the presence of MgCl2 (Sigma Aldrich) to avoid residual DNA, and ultraconcentrated by centrifugation 1 h at 22000 rpm at 4° C. Each vector pellets are resuspended in 70 μl PBS-Lactose (40 mg/L), pooled, 30 μL aliquoted and stored at −70° C.±10° C. Hence for each production, 210 mL of harvest clarified bulk is finally resuspended in 420 μL of PBS lactose.

### Example 4

### Quantification of Lentiviral Vectors by Flow Cytometry

For the quantification of infective particles, HEK 293T cells were seeded in 24-well plates (BD Falcon) at a density of 1×10⁵ cells per well in complete medium containing 10% FBS and incubated for 4 h to adhere. The cells were transduced by replacing the medium with 300 μl of dilutions 1/100, 1/300 and 1/900 of viral samples in complete medium, followed by incubation at 37° C., 5% CO₂ for 2 h. After adsorption, 1 mL of complete medium was added to each well. At 72 h posttransduction, the cells were trypsinized and resuspended in 300 μL of complete medium, and the percentage of cells expressing GFP was determined with an ACCURI flow cytometer (BD Biosciences). Two sets of three dilutions were performed for each sample tested. The values corresponding to a percentage of transduced cells less than 30% were used to calculate the approximate number of transducing units (TU) present in the viral suspension.

$$Titer ( T U / mL ) = \frac{( \% transduced cells \times {1.10}^{5} )}{100 } \times \frac{1000 \times dilution factor}{300}$$

### Example 5

### Lentiviral Vector Titration

HEK 293T cells were seeded in 6-well plates (BD Falcon) in culture medium and incubated for 4 h at 37° C., 5% CO2 in moist atmosphere. Cells were transduced with 3 successive dilutions ( 1/800, 1/1600 and 1/3200) of ultracentrifuged lentiviral vector. 72 h post-incubation, cells are harvested and transduced HEK 293T cell pellets are produced. Total genomic DNA from transduced cell-pellets is extracted using a method based on QIAGEN QIAamp DNA mini kit handbook. Proviral quantification is performed using Taqman qPCR. The amplification is performed with the Master Mix (Fermentas Thermo Scientific), the Flap A (CCCAAGAACCCAAGGAACA; SEQ ID NO:38) and Flap S (AGACAA GATAGAGGAAGAGCAAAAC; SEQ ID NO:39) primers and Lenti TM probe (6FAM-AACCATTAGGAGTAGCACCCACCAAGG-BBQ; SEQ ID NO:40). Normalization is performed with the quantification of the actin gene (same Mix, Actine A-CGGTGAGGATCTTCATGAGGTAGT-; SEQ ID NO:41, Actine S-AACACCCCAGCCATGTACGT-; SEQ ID NO:42 primers and Humura ACT TM probe-6FAM-CCAGCCAGGTCCAGACGCAGGA-BBQ-; SEQ ID NO:43. Both reactions are achieved on MasterCycler Ep Realplex S (Eppendorf, 2 min at 50° C., 10 min at 95° C. and 40 cycles of 15 seconds at 95° C. and 1 min at 63° C.). The analysis is performed on MasterCycler Ep Realplex Software.

### Example 6

### Pseudotyped VSV Alagoas and Viral Hemoragic Septicemia Virus FR-L59X G Lentivector Productions

Codon optimized genes of VSV Alagoas and viral Hemoragic septicemia virus FR-L59X G proteins were synthesized, and cloned between the BamH1 and EcoR1 sites of the pThV-plasmid, encompassing the WPREm sequence or not, hence generating the pThV-VSV.G(ALAGOAS-CO), pThV-VSV.G(ALAGOAS-CO)-WPREm, pThV-VSV.G(FR-L59X-CO) and the pThV-VSV.G (FR-L59X-CO)-WPREm vectors.

Codon optimized genes were generated for the VSV Alagoas and viral Hemoragic septicemia virus FR-L59X G proteins. The genes were cloned between the BamH1 and EcroR1 sites of the pThV plasmid, encompassing or not the WPREm.

The nucleic acid sequence of the Codon optimized Alagoas gene is:

(SEQ ID NO: 1)
atgacccccgccttcatcctgtgcatgctgctggccggctctagctgggc
caagttcaccatcgtgttcccccagagccagaagggcgactggaaggacg
tgccccccaactaccggtactgccccagcagcgccgatcagaactggcac
ggcgacctgctgggcgtgaacatccgggccaagatgcccaaggtgcacaa
ggccattaaggccgatggctggatgtgtcacgccgccaaatgggtgacca
cctgcgactaccggtggtacggcccccagtacatcacccacagcatccac
agcttcatccccaccaaggcccagtgcgaggaaagcatcaagcagaccaa
agagggcgtctggatcaaccccggcttcccccccaagaactgcggctacg
ccagcgtgtccgacgccgagagcatcatcgtgcaggccaccgcccacagc
gtgatgatcgacgagtacagcggcgattggctggactctcagttccccac
aggccggtgtaccggcagcacctgcgagacaatccacaacagcaccctgt
ggtacgccgactaccaggtcacaggcctctgcgacagcgccctggtgtcc
accgaagtgaccttctacagcgaggacggcctgatgacctctatcggccg
gcagaacaccggctacagaagcaactacttcccctacgagaagggcgctg
ctgcctgccggatgaagtactgcacccacgagggcattcggctgcccagc
ggcgtgtggttcgagatggtggacaaagagctgctggaaagcgtccagat
gcccgagtgccctgccggcctgaccatcagcgcccccacccagaccagcg
tggacgtgtccctgatcctggacgtggagcggatgctggactacagcctg
tgccaggaaacctggtctaaggtccacagcggcctgcccatctcccccgt
ggacctgggctatatcgcccccaagaatcctggcgccggacccgccttta
ccatcgtgaacggcaccctgaagtacttcgacaccagatacctgcggatc
gacatcgagggccccgtgctgaagaagatgaccggcaaggtctccggcac
ccccaccaagcgggagctgtggaccgagtggttcccttacgacgacgtgg
agatcggccccaacggcgtgctgaaaacccccgagggctacaagttcccc
ctgtacatgatcggccacggcctgctggactccgacctgcagaaaaccag
ccaggccgaggtgttccaccacccccagatcgccgaggccgtgcagaagc
tgcccgacgacgagacactgttcttcggcgacaccggcatcagcaagaac
cccgtggaagtgatcgagggctggttcagcaactggcggagcagcgtgat
ggccatcgtgtttgccatcctgctgctggtcattaccgtgctcatggtcc
ggctgtgcgtggccttccggcacttctgctgccagaagcggcacaagatc
tacaacgacctggaaatgaaccagctgcggagatgatga.

The encoded amino acid sequence of Alagoas is:

(SEQ ID NO: 2)
MTPAFILCMLLAGSSWAKFTIVFPQSQKGDWKDVPPNYRYCPSSADQNWH
GDLLGVNIRAKMPKVHKAIKADGWMCHAAKWVTTCDYRWYGPQYITHSIH
SFIPTKAQCEESIKQTKEGVWINPGFPPKNCGYASVSDAESIIVQATAHS
VMIDEYSGDWLDSQFPTGRCTGSTCETIHNSTLWYADYQVTGLCDSALVS
TEVTFYSEDGLMTSIGRQNTGYRSNYFPYEKGAAACRMKYCTHEGIRLPS
GVWFEMVDKELLESVQMPECPAGLTISAPTQTSVDVSLILDVERMLDYSL
CQETVVSKVHSGLPISPVDLGYIAPKNPGAGPAFTIVNGTLKYFDTRYLR
IDIEGPVLKKMTGKVSGTPTKRELWTEWFPYDDVEIGPNGVLKTPEGYKF
PLYMIGHGLLDSDLQKTSQAEVFHHPQIAEAVQKLPDDETLFFGDTGISK
NPVEVIEGWFSNWRSSVMAIVFAILLLVITVLMVRLCVAFRHFCCQKRHK
IYNDLEMNQLRR.

The nucleic acid sequence of the Codon optimized FR-L59X gene is:

(SEQ ID NO: 3)
atggaatggaatacctttttcctggtcatcctgatcatcatcatcaagag
caccacctcccagatcacccagcggcctcccgtggagaacatcagcacct
accacgccgactgggacacccctctgtacacccaccccagcaactgccgg
gaggacagcttcgtgcccatcagacccgcccagctgcggtgcccccacga
gttcgaggacatcaacaagggcctggtgtccgtgcccacccagatcatcc
acctgcccctgagcgtgaccagcgtgtccgccgtggcctctggccactac
ctgcacagagtgacctaccgcgtgacctgcagcaccagctttttcggcgg
ccagaccatcgagaaaaccatcctggaagccaagctgtcccggcaggaag
ccgtcaacgaggccagcaaggaccacgagtaccctttcttccccgagccc
agctgcatctggatgaagaacaacgtgcacaaggacatcacccactacta
caagacccccaagaccgtgtccgtggacctgtacagccggaagttcctga
accccgacttcatcgagggcgtgtgcaccacaagcccctgccagacccac
tggcagggcgtgtactgggtcggcgccacacccaccgcccactgccccac
ctccgagacactggaaggccacctgttcacccggacccacgaccatcggg
tggtcaaggccatcgtggctggccaccacccttggggcctgaccatggcc
tgcaccgtgaccttttgcggcaccgactggatcaagaccgacctgggcga
cctgatcaaagtgatcggccagggcggcgagaagaagctgacccccaaaa
agtgcgtgaacaccgacatccagatgagaggcgccaccgacgacttcagc
tacctgaaccacctgatcaccaacatggcccccagaaccgagtgcctgga
cgcccacagcgacatcaccgccagcggcaagatcagcagcttcctgctgt
ccaagttccggcccagccaccccggacctggcaaggcccactatctgctg
gacggccagatcatgcggggcgactgcgactacgaggccgtggtgtccat
caactacaacagcgcccagtacaagaccgtgaacaacacctggaagtcct
ggaagcggatcggcaacaacaccgacggctacgacggcatgatcttcggc
gacaagctggtcatccccgacatcgagaagtaccagagcatctacgacag
cggcatgctggtgcagcggaacctggtggaggtgccccacctgagcatcg
tgttcgtgagcaacaccagcgacctgagcaccaaccacatccacaccaac
ctgatccccagcgactggtccttccactggtccctgtggcccagcctgag
cggcatgggcatcgtgggcggagcctttctgctgctggtgctgtgctgct
gctgcaaggccagcccccccatccccaactacggcatccccatgcagcag
ttcagccggaaccagatggtctgatga.

The encoded amino acid sequence of FR-L59X is:

(SEQ ID NO: 4)
MEWNTFFLVILIIIIKSTTSQITQRPPVENISTYHADWDTPLYTHPSNCR
EDSFVPIRPAQLRCPHEFEDINKGLVSVPTQIIHLPLSVTSVSAVASGHY
LHRVTYRVTCSTSFFGGQTIEKTILEAKLSRQEAVNEASKDHEYPFFPEP
SCIWMKNNVHKDITHYYKTPKTVSVDLYSRKFLNPDFIEGVCTTSPCQTH
WQGVYVVVGATPTAHCPTSETLEGHLFTRTHDHRWKAIVAGHHPWGLIMA
CTVTFCGTDWIKTDLGDLIKVIGQGGEKKLTPKKCVNTDIQMRGATDDFS
YLNHLITNMAPRTECLDAHSDITASGKISSFLLSKFRPSHPGPGKAHYLL
DGQIMRGDCDYEAVVSINYNSAQYKTVNNTWKSWKRIGNNTDGYDGMIFG
DKLVIPDIEKYQSIYDSGMLVQRNLVEVPHLSIVFVSNTSDLSTNHIHTN
LIPSDWSFHWSLWPSLSGMGIVGGAFLLLVLCCCCKASPPIPNYGIPMQQ
FSRNQMV.

Lentivector batches were produced by tri-transfection of HEK 293T cells with a proviral plasmid (CMV-GFP), a packaging plasmid and the plasmid encompassing either the Indiana, New Jersey, Alagoas or FR-L59X G pseudotyping proteins, with or without the WPREm sequence. Those various batches were produced in triplicate. They were used to transduce HEK 293T cells and for each batches, titers (TU/mL) were evaluated by GFP monitoring (FACS analysis). More precisely, HEK 293T cells were seeded in 24-well plates at a density of 10⁵ cells per well in complete medium containing 10% FBS. Wells of 10⁵ cells were transduced by replacing the culture medium with 300 μl of the dilution of the various viral batches triplicates which allowed a percentage of transduced cells included between 5 and 30%. The cells were then incubated 2 h at 37° C., 5% CO2 and 1 ml of complete medium was added per well. 72 h post transduction, the cells were trypsinized and resuspended, and the GFP MFI was measured with a FACScalibur flow cytometer, using the FL1 channel.

### Example 7

### Cross Neutralization Assays

Mice C57Bl/6 mice (haplotype H2b, between 12 and 23 weeks old) were intraperitoneally injected with the viral particles pseudotyped with the VSV-G serotypes (Indiana, New Jersey, Isfahan, Cocal SVCV and Isfahan, 6 mice per group, 450 μL/mouse). 4 weeks later, the mice were boosted with the same particles (500 μL/mouse). A first retro orbital blood collection (in Capiject tubes) is done 15 days post boost, and a second 21 days post boost. The blood is centrifuged 6 min at 3500 rpm and the serum is collected and kept at −20° C.

Transduction assays were made in presence of various dilutions of these sera.

### Example 8

### Cellular Immunogenicity

C57Bl/6j mice were immunized with 10⁶ TU of pseudotyped lentiviral vectors bearing 6 glycoproteins derived from other enveloped viruses from Rhabdoviridae family: Indiana, New Jersey, Cocal, SVCV (Spring Viremia Carp Virus), Isfahan and Alagoas. 15 days after immunization, splenocytes were isolated from the immunized and control mice spleens and the specific T-cell responses were monitored in mice splenocytes by IFN-γ ELISPOT.

Ninety-six-well tissue culture plates (Millipore) were coated overnight at 4° C. with 50 μl/well of 5 μg/ml anti-mouse IFNγ mAb (Mouse IFNγ Elispot pair; BD Biosciences Pharmingen). The plates were washed three times with 200 μl DPBS/well and blocked with 200 μl/well of DPBS/10% fetal bovine serum for 2 h at 37° C. Splenocytes were added to the plates in triplicate at 1×10⁶ cells/well and stimulated with 2 μg/ml of stimulatory peptides (specific to the antigen), concanavalin A (1.5 μg/ml; source), or culture medium alone. The plates were incubated for 24 h at 37° C. and then rinsed three times with 200 μl/well of DPBS/0.05% Tween 20 and three times with 200 μl/well of distilled water. For detection, 50 μl/well of 2 μg/ml anti-mouse IFNγ-biotinylated monoclonal antibody (BD Pharmingen) were added for 2 h at room temperature. Plates were washed (3 times with DPBS/0.05% Tween) and 100 μl/well of streptavidin-alkaline phosphatase (Roche) diluted 1:2000 in Dulbecco's PBS-10% SVF for 80 min at room temperature. After washing the plates (3 times with DPBS/0.05% Tween and 3 times with DPBS), spots (IFNγ-secreting cells) were revealed by adding 100 μl/well of BCIP/NBT solution (Sigma). Plates were incubated for 20 min at room temperature until blue spots developed and then thoroughly washed with running tap water and air-dried for 24 h. Finally, the spots were counted using an AID Reader.

1. A combination of compounds for sequential administration to a mammalian host comprising:
(i) a composition comprising at least 10⁶ TU/ml of lentiviral vector particles pseudotyped with a first viral G protein, for a first administration; and
(ii) a composition comprising at least 10⁶ TU/ml of lentiviral vector particles pseudotyped with a different, second viral G protein, for a second administration;
wherein the first viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G proteins.

2. (canceled)

3. The combination of compounds of claim 1, wherein the second viral G protein is selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G proteins.

4. (canceled)

5. The combination of compounds of claim 1, wherein the second viral G protein is of the VSV Indiana strain.

6. The combination of compounds of claim 1, wherein the second viral G protein is of the VSV New Jersey strain.

7. The combination of compounds of claim 1, wherein the second viral G protein is of the Cocal strain.

8. The combination of compounds of claim 1, wherein the second viral G protein is of the SVCV strain.

9. The combination of compounds of claim 1, wherein the second viral G protein is of the Piry strain.

10. The combination of compounds of claim 1, wherein the second viral G protein is of the Isfahan strain.

11-16. (canceled)

17. The combination of compounds of claim 1, wherein the lentiviral vector particles for the first or second administration comprise a nucleic acid comprising a functional lentiviral DNA flap.

18. The combination of compounds of claim 17, wherein the functional lentiviral DNA flap is an HIV-1 DNA flap.

19. (canceled)

20. (canceled)

21. A method for priming and subsequently boosting an immune response in a mammalian host comprising sequentially administering the combination of compounds of claim 1, wherein the first administration and second administration are administered at different times to a mammalian host.

22. A composition comprising:
(i) at least 10⁶ TU/ml of unconcentrated lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G proteins; or
(ii) at least 10⁸ TU/ml of unconcentrated lentiviral vector particles pseudotyped with a G protein selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G proteins.

23. (canceled)

24. An expression vector comprising a nucleotide sequence comprising a codon optimized G protein gene selected from Yug Bogdanovac virus, VSIV-3 86 Agulhas NegrasB, VSIV-2 86 MaipuE, Vesicular stomatitis Alagoas virus, VSIV-2 98 ParanaE, VSIV-3 95 Minas GeraisB, Maraba virus, Piry virus, Perinet virus, Snakehead rhabdovirus, Kimberley virus CS368, Jurona virus, West Caucasian Bat virus, Lagos bat 8619NGA virus, and Pike Fry rhabdovirus G protein genes.

25-27. (canceled)


SEQUENCE LISTING (43 sequences)

SEQ ID NO: 1 - DNA, 1539 bp, VESICULAR STOMATITIS ALAGOAS VIRUS
atgacccccgccttcatcctgtgcatgctgctggccggctctagctgggccaagttcaccatcgtgttcccccagagccagaagggcgactggaaggacgtgccccccaactaccggtactgccccagcagcgccgatcagaactggcacggcgacctgctgggcgtgaacatccgggccaagatgcccaaggtgcacaaggccattaaggccgatggctggatgtgtcacgccgccaaatgggtgaccacctgcgactaccggtggtacggcccccagtacatcacccacagcatccacagcttcatccccaccaaggcccagtgcgaggaaagcatcaagcagaccaaagagggcgtctggatcaaccccggcttcccccccaagaactgcggctacgccagcgtgtccgacgccgagagcatcatcgtgcaggccaccgcccacagcgtgatgatcgacgagtacagcggcgattggctggactctcagttccccacaggccggtgtaccggcagcacctgcgagacaatccacaacagcaccctgtggtacgccgactaccaggtcacaggcctctgcgacagcgccctggtgtccaccgaagtgaccttctacagcgaggacggcctgatgacctctatcggccggcagaacaccggctacagaagcaactacttcccctacgagaagggcgctgctgcctgccggatgaagtactgcacccacgagggcattcggctgcccagcggcgtgtggttcgagatggtggacaaagagctgctggaaagcgtccagatgcccgagtgccctgccggcctgaccatcagcgcccccacccagaccagcgtggacgtgtccctgatcctggacgtggagcggatgctggactacagcctgtgccaggaaacctggtctaaggtccacagcggcctgcccatctcccccgtggacctgggctatatcgcccccaagaatcctggcgccggacccgcctttaccatcgtgaacggcaccctgaagtacttcgacaccagatacctgcggatcgacatcgagggccccgtgctgaagaagatgaccggcaaggtctccggcacccccaccaagcgggagctgtggaccgagtggttcccttacgacgacgtggagatcggccccaacggcgtgctgaaaacccccgagggctacaagttccccctgtacatgatcggccacggcctgctggactccgacctgcagaaaaccagccaggccgaggtgttccaccacccccagatcgccgaggccgtgcagaagctgcccgacgacgagacactgttcttcggcgacaccggcatcagcaagaaccccgtggaagtgatcgagggctggttcagcaactggcggagcagcgtgatggccatcgtgtttgccatcctgctgctggtcattaccgtgctcatggtccggctgtgcgtggccttccggcacttctgctgccagaagcggcacaagatctacaacgacctggaaatgaaccagctgcggagatgatga

SEQ ID NO: 2 - protein, 511 aa, VESICULAR STOMATITIS ALAGOAS VIRUS
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 3 - DNA, 1527 bp, VIRAL HEMORAGIC SEPTICEMIA VIRUS FR-L59X
atggaatggaatacctttttcctggtcatcctgatcatcatcatcaagagcaccacctcccagatcacccagcggcctcccgtggagaacatcagcacctaccacgccgactgggacacccctctgtacacccaccccagcaactgccgggaggacagcttcgtgcccatcagacccgcccagctgcggtgcccccacgagttcgaggacatcaacaagggcctggtgtccgtgcccacccagatcatccacctgcccctgagcgtgaccagcgtgtccgccgtggcctctggccactacctgcacagagtgacctaccgcgtgacctgcagcaccagctttttcggcggccagaccatcgagaaaaccatcctggaagccaagctgtcccggcaggaagccgtcaacgaggccagcaaggaccacgagtaccctttcttccccgagcccagctgcatctggatgaagaacaacgtgcacaaggacatcacccactactacaagacccccaagaccgtgtccgtggacctgtacagccggaagttcctgaaccccgacttcatcgagggcgtgtgcaccacaagcccctgccagacccactggcagggcgtgtactgggtcggcgccacacccaccgcccactgccccacctccgagacactggaaggccacctgttcacccggacccacgaccatcgggtggtcaaggccatcgtggctggccaccacccttggggcctgaccatggcctgcaccgtgaccttttgcggcaccgactggatcaagaccgacctgggcgacctgatcaaagtgatcggccagggcggcgagaagaagctgacccccaaaaagtgcgtgaacaccgacatccagatgagaggcgccaccgacgacttcagctacctgaaccacctgatcaccaacatggcccccagaaccgagtgcctggacgcccacagcgacatcaccgccagcggcaagatcagcagcttcctgctgtccaagttccggcccagccaccccggacctggcaaggcccactatctgctggacggccagatcatgcggggcgactgcgactacgaggccgtggtgtccatcaactacaacagcgcccagtacaagaccgtgaacaacacctggaagtcctggaagcggatcggcaacaacaccgacggctacgacggcatgatcttcggcgacaagctggtcatccccgacatcgagaagtaccagagcatctacgacagcggcatgctggtgcagcggaacctggtggaggtgccccacctgagcatcgtgttcgtgagcaacaccagcgacctgagcaccaaccacatccacaccaacctgatccccagcgactggtccttccactggtccctgtggcccagcctgagcggcatgggcatcgtgggcggagcctttctgctgctggtgctgtgctgctgctgcaaggccagcccccccatccccaactacggcatccccatgcagcagttcagccggaaccagatggtctgatga

SEQ ID NO: 4 - protein, 507 aa, VIRAL HEMORAGIC SEPTICEMIA VIRUS FR-L59X
MEWNTFFLVILIIIIKSTTSQITQRPPVENISTYHADWDTPLYTHPSNCREDSFVPIRPAQLRCPHEFEDINKGLVSVPTQIIHLPLSVTSVSAVASGHYLHRVTYRVTCSTSFFGGQTIEKTILEAKLSRQEAVNEASKDHEYPFFPEPSCIWMKNNVHKDITHYYKTPKTVSVDLYSRKFLNPDFIEGVCTTSPCQTHWQGVYWVGATPTAHCPTSETLEGHLFTRTHDHRVVKAIVAGHHPWGLTMACTVTFCGTDWIKTDLGDLIKVIGQGGEKKLTPKKCVNTDIQMRGATDDFSYLNHLITNMAPRTECLDAHSDITASGKISSFLLSKFRPSHPGPGKAHYLLDGQIMRGDCDYEAVVSINYNSAQYKTVNNTWKSWKRIGNNTDGYDGMIFGDKLVIPDIEKYQSIYDSGMLVQRNLVEVPHLSIVFVSNTSDLSTNHIHTNLIPSDWSFHWSLWPSLSGMGIVGGAFLLLVLCCCCKASPPIPNYGIPMQQFSRNQMV

SEQ ID NO: 5 - protein, 559 aa, YUG BOGDANOVAC VIRUS
MISSTLILVIISAHAFCDMIIPFPDVTTTSWKPVLRGEHHCPASNDLDMAGGLSTLKMNVKIPSGVVGSKSDGYLCHGAKWVTTCDYRWYGAKYITHSLHPLRPSTSQCFDAIKAYREGTLLSPGFPPESCGWNSVTDSELLSIQITPHHSGVDDYRGVWIDSMFPKGECDQRICDTVQEHSIWIAANNVSSACSIAFKQLEGYFYYRNSGIQPNKDGTFFHSSHHPNSPMSSCCRIKYCNQEGLRLHTGEWIGVAWNTKIRDVTLDSYTDTCPGGTEVKSTIGSSPTRVVAWEMERIMDFALCQNVWDKVNRGEQLSPLDLSYLSSRAPGKGLAYTIINETLHVAHVRYIRTWIKGPVLKEIKGRRGSSSAAEDTLWIQWFPFGDNQIGPNGLLKSNGTFKFPFYLVGVGALDEDLIEMANADPVDHLQRVDAETHMRGDEELFFGDTGVSKNPIESVEGWFSNWISGLFNISIIVLCVLSVLIVFKSVITLIRVVRRRRRPRAEEDVELNNMNPRPQTRQPVGAPNIIPGAWGIQPSHGRGVRQSQFVKRSALNIVT

SEQ ID NO: 6 - protein, 623 aa, WONGABEL VIRUS
MSFLLAIIIFFFRTQDSNGYVVQSGIGGIRESKVNILGQVVPYSTKFKHSVLKSQWDYRHDKGHRTVLPTNCHATWNDITPSNMRCPRRKIIGKDGLYNTYIGDFWHPHTDKGSEVKGFICQKTKWVSTCIETWYFSTTKETQIDEVPITKEDCLAAITLVDSGEYIEPFFPPHVCSWASTNKNSKEFVTVHEHSVVLDIYENKLMDPIFLAGKCFDKVCKTIHRNVLWVEANDNERDDFCVATAWEYSHVFADIDIDHNNNHPIYSIGKTIDSEIYGPRDLADACIIKICGIPGIRFSHGEWWGIKTLSDRIPLEDIIIKCHNGTSVGFVHNIWTPSELVGEITYRDHKCLDVLSSFLGQRKINPYELSYLVQDFPGEGPAYRIMKQYTGNNKTKATFRLQTKTCRYHVAYIDKLTFDPENGTDEVYKLGTWGNGRTVILNSTEVGINPTYINKSFDWEPLETFNGLMRFGAELVLPQAVYTDHPNITNLLEDYEISLIGHPKEIFEPEQDELSQVYKFYERSNSTNVVGLASNFVKTIGRSIGNFFGGTKNLIWWLVTVALSTIGTYIAYKLGLFKFLGRILFQGSESKEDKRVSNIYEEPLKLGGRRSHLVKNPFFDNGI

SEQ ID NO: 7 - protein, 526 aa, MOUSSA VIRUS ISOLATE C23
MRTLVIWFLINVTMAFAKPPGSASLSLGLYWVPRIDNNTWKSVHTTNLVCPSFVGSVLPEMEESFEIDIQVPKHSQTTSHQGGYLCYGFSFSVVCEEGFWGGQKVTEHTFTHLVSSEECLKAIEDKKSGEYRPPHTPVSECGWMQTNTKTLRFVTLEEHPVLFDPYTVNFVDGLFEKTLCNQRICPTVHANTIWIGDNEPKKDCPPTENEKAVLYVEKQNVVPVVWVKLTGGTVYKLDRACTMTYCDIDGVRMEDGHWFAGVNLTQYVRRDCDKGMDITFDTLASLSLLTKIELEHVQDRMECLDAVQDLRAGGKVTYAKLSKLQPKRGGLFHVYRINKGTLEYTMGRYEGLTSLITNIPFVIGKNQKDEKVQLHHVPSGDNSTLSSYNGVHMFLNGTVIIPEMELYKLRYSETLLYEHLLGKMKHPSAKQRERMGLTPDDDKRTTNKSLNIGEWFSSFWSHLVGKIVSILGTALAIFLILYICWTCLKIQIKRVSDKNRVDQMEMQILSKARAPEVRPTLSGPIW

SEQ ID NO: 8 - protein, 525 aa, WEST CAUCASIAN BAT VIRUS
MASYFALVLNGISMVFSQGLFPLYTIPDHLGPWTPIDLSHLHCPNNLYTDASYCTTEQSITYTELKVGSSVSQKIPGFTCTGVRTESVTYTNFVGYVTTTFKKKHFPPKSRDCREAYERKKAGDPRYEESLAHPYPDNSWLRTVTTTKDSWVIIEPSVVELDIYTSALYSPLFKDGTCSKSRTYSPYCPTNHDFTIWMPESENIRSACNLFSTSRGKLVRNRTSTCGIIDERGLFRSVKGACKISICGRQGIRLVDGTWMSFRYSEYLPVCSPSQLINTHDIKVDELENAIVLDLIRRREECLDTLETILMSGSVSHRRLSHFRKLVPGSGKAYSYINGTLMESDAHYIKVENWSEVIPHKGCLMVGGKCYEPVNDVYFNGIIRDSNNQILIPEMQSSLLREHVDLLKANIVPFRHPMLLRSFTSDTEEDIVEFVNPHLQDTQKLVSDMDLGLSDWKRYLLIGSLAVGGVVAILFIGTCCLRCRAGRNRRTIRSNHRSLSHDVVFHKDKDKVITSWESYKGQTAQ

SEQ ID NO: 9 - protein, 655 aa, KIMBERLEY VIRUS ISOLATE CS368
MSSEIKMLIELMLFSILSCVISQRVYNFPFNCTEPERIKDYQIKCPIRQNEVSLEAHHVEVDEKIEKICRPQIKDDDHIEGYICREQHWTTKCTETWYFSTEIEYTIKETVPNQADCQKELEKLKRGISIPPYYPPAGCFWNMAQSEKITFVVLVPHKVLQNPYDMKLYDPGFIEKCDVKKAKTKGCKMKDITGLWVTNNDGKNTSEHCNKDHWECIGIKSFRSELNLHDRLWESPELGIMKLNKACKKNFCGYKGVILEDGEWWGYTNVADSEIEYVHLNNCDDSRLPGFRIHQDRTEFEEFDIKAEMENERCMNTLSKILNKENLNFVDMSYLSPSRPGRDYAYLFEQVSWDETFCLTWPDSRKSKNCKVDWKVHKNAGLVTKKHGAIGTYYRSMCMYYPIEDTNKDGILQKDELKDKGIPGKNRYRTLKRSKNDYGEDSEFNITYNGMVVVNESFHMAVKSIYDGTEDYNSLLKFEVSEFDKIDLNEAYKEEENKWNDIDLTPVSSVNRSRSDIIKEVEKGGRKIISAVTGWFTGLAKTVRWTIWGIGSIVTIYAIWKLKKMITKKNKEDKNLVNHNELNEAFEMSKDVERGRVETWIRKNKGKEEGIYEQVSDIEDNVSKYERGVHASKGGDKMNVYSPHGKNGKKGFFNH

SEQ ID NO: 10 - protein, 618 aa, FLANDERS VIRUS
MSYLIVKVVILFLVGIDKQVLSWTHDSGRSFVRQYHDPNWFDQTMVYPIECNSTWQEVNTLNLRCPKSLKIDPKNKLNFDLGTVYHPLPSSRYVVNGYICHKQTWISKCEETWYFSTTETNKIENVPITPEDCREAVTIYEMGEYVNPFFPPFYCSWSSTQIDKKTFVIVEPHIVKEDIYNKTFIDPFFLNGYCDQLPCKTIHPDVLWVPQELQKRKDLCNKGTWETGKVFGVLEEKLYQNGYLKDNRFGIDEQWIRSSIYGLRSLVGSCYRGVCRQFGIRFKTGEWWGLEGKDVTGWIKQIIPRCQENQYVSFHHDNSDENIAEAQLVARELVCEEFLGRAKGGDLISPFDLNYLLPLNPGLGPSYRAFKRILKKDSHGGSSPQFRLEKRDCIYSVVHNVTEKVNITNNKLAIGQLFDGSYVYINESEFSRPDYLNNSDNASRDDWFLLSLNGMIKYGNSVYLPHGVSTGLSGIQDIVERGTLMLLDHPKSIAISNQMDLAKNIYTSYFQMNTTSIGSKIENMIIRAKNAVSSYFSQLTNIAWWIGTGILGLLGFIVIKRFHLIQLICGKKHRNGKIKKKNNKLNNDDQEAHVYDTIFNTPKTPPHGKGTGVKYFDY

SEQ ID NO: 11 - protein, 512 aa, VESICULAR STOMATITIS VIRUS ISOLATE VSIV-2 86MAIPUE
MNFILLILAVLPISNYAKFSIVFPQSQKGNWKNVPASYHYCPSSSDQNWHNDLIGITMKVKMPKTHKAIQADGWMCHAAKWITTCDFRWYGPKYITHSIHSIQPTSEQCKESIKQTKQGTWISPGFPPQNCGYATVTDSVAVIVQATPHHVLVDEYTGEWIDSQFPNGKCEAEECETIHNSTVWFSDYKVTGLCDATLVDTDITFFSEDGKKESLGKPNTGYRSNYFAYEKGEKVCKMNYCKHSGVRLPSGVWFEFADQDAYTAAKFPECPVGATISAPTQTSVDVSLILDVERILDYSLCQETWSKIRSKQPVSPVDLSYLAPKNPGTGPAFTIINGTLKYFETRYIRIDIDNPIIPKMVGKIGGSQTERELWTEWYPYEDVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVIARIVITVRSRYQGASNKRIYNDIEMSRFRK

SEQ ID NO: 12 - protein, 511 aa, VESICULAR STOMATITIS VIRUS ISOLATE VSIV-3 86AGULHAS NEGRASB
MNPAFVLCMLLAGSSWAKFAIVFPQSQKGEWKDVPPNYRYCPSSADQNWHGDLLGVNIRAKIPKAHKAIKADGWMCHAAKWITTCDYRWYGPQYITHSIHSFVPTQAQCEESIKQTKEGVWINPGFPPKNCGYASISDAESVIVQATAHTVMIDEYSGDWLDSQFPTGRCKETICETIHNSTLWYADYKVTGLCDSALVSTEVTFYSEDGLVTSIGRQNTGYRSNYFPYEKGAAACRMKYCTYDGIRLPSGVWFEMVDKELLDSAQMPECQAGLTISAPTQTSVDVSLILDVERMLDYSLCQETWSKVHNGQPISPVDLGYIAPKNPGAGPAFTIINGTLKYFDTRYLRIDIDGPVLKKMAGKISGTLTERELWTEWFPYEDVEIGPNGVLKTPEGYKFPLYMIGHGLLDADLQKTSQAEVFHHPQIAEAIKKLPDDETLFFGETGISKNPVELIEGWFSNWRSSVMAVVFAIALLVVLVLIVRIIVAFRHSCCRRQRKIYDDLEMNQLRK

SEQ ID NO: 13 - protein, 512 aa, MARABA VIRUS
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNYFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 14 - protein, 512 aa, VSIV-2 98 PARANAE
MNFLLLFLVTIPAYSYAKFSIVFPQSQKGNWKNVPATYHYCPSSSDQNWHNDLIGIPMKVKMPKTHKAIQADGWMCHAAKWITTCDFRWYGPKYITHSIHSIQPTSEQCKESIKQTKQGTWINPGFPPQNCGYASVSDSVAVIVQATPHHVLVDEYTGEWIDSQFPNGKCKAEECETIHNSTVWFSDYKVTGLCDATLVDTDITFFSEDGKKESLGKPNTGYRSNYFAYEKGEKVCKMNYCKHAGVRLPSGVWFEFADQDAYTAAKFPECPVGATISAPTQTSVDVSLILDVERILDYSLCQETWSKIRSKQPVSPVDLSYLAPKNPGTGPAFTVINGTLKYFETRYIRIDIDNPIIPKMVGKVGGSHTERELWTEWYPYEDVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILFCVVARIVIAVRYRYQGASKKRIYNDVEMSRFRK

SEQ ID NO: 15 - protein, 511 aa, VSIV-3 95 MINAS GERAISB
MNLAVVLCMLLAGSSWAKFTIVFPQSQKGEWRDVPSNYRYCPSSADQSWHGDLLGVNIRAKIPKAHKAIKADGWMCHAAKWITTCDYRWYGPQYITHSIHSFVPTQAQCEESIKQTKEGVWINPGFPPKNCGYASINDAESVIVQATAHTVMIDEYSGDWLDSQFPTGRCKGTICETIHNSTLWHADYKVTGLCDAALVSTEVTFYSEDGLVTSIGRQNTGYRSNYFPYEKGAGACRMKYCTHDGIRLPSGVWFEMVDKELLNSSQMPECPAGLTITAPTQTSVDVSLILDVERMLDYSLCQETWSKVHNGQPISPVDLGYIAPKNPGAGPAFTIINGTLKYCDTRYLRIDIDGPVLQKMTGKVSGTPTERELWTEWFPYEDVEIGPNGVLRTQEGYKFPLFMIGHGLLDADLQKTSRAEVFHHPQIAEAVKKLPDDETLFFGETGISKNPVELIEGWFSNWRSSVMAVIFAIVSLVLLGLIVRVIVAFRNSCCQRKRKVYNDLEMNRLRN

SEQ ID NO: 16 - protein, 524 aa, JURONA VIRUS
MESLPFSALLAVLSITLCDSAIPIFFPSEPQLEWKPVLPGSRYCPQSNEMSLDPDLKKSTISVKVPIGVTPSKSDGYLCHGAKWVSTCDFRWYGPKYITHSIHNLRPTTNDCEDAIKKYEAGTLINPGFPPDSCAYATVTDSEHLVILITPHHVGVDDYRGAWVDDSFPSGVCETNQCDTTHNSSIWIPKTKTRHNICSQTFANLSVTISYREGGAMKGADMVFHSKYHPHMVGGHICKMNFCNKQGLRLQNEEWIEIPSGTKVGNQDLMNLFSDCKSGLEVRSTLRSEGANTLTWETQRLLDYALCQNTWDKFDNQGAVSALDLSYLAARAPGKGVAYTMINGTLHSAPTRYVRMWIESPSMEELKAKKESSSGVETSIWNQWFPFKGGEIGPNGLIKAGNKYKFPLYLVGMGMLDDEINALELGGPIDHPQRAHAQAVLGDEETLFFGDTGVGKNPVELITGWFSGWKETIMAVVAIFLLVIVLYGVLRCCPTICVLCKRKSRHRTKDMEMQYIPNNQRHWR

SEQ ID NO: 17 - protein, 525 aa, PERINET VIRUS
MSSKIVLAAICLCSVQYVACSFQIVFPEFNNAAWLPYLKTSRYCPQSAEMEFERRVSTTLLSADVPIGVTPTKSDGYLCHAAKWVTTCDFRWYGPKYVTHSIHDLTPAQVDCHEALARYKAGTLFNPGFPPASCGYATITDSEQKVVMITPHHVGIDDYRGKWIDPIFPGGECTTNYCETLHNSSVWLPADEKIVDICAQTFRKIKVTATYPSEGAVTKETISLHSAYHPHVPGTGICRMTYCSKEGLRLPNGEWLGIFYDNRIKTTDVRTVFPACPDGLEVKSTLNSDGANTIAWETQRMLDYALCQSTWDKVQNKEPLSAVDLSYLSARSPGKGLAYTVINGTLHFAHVRYVRTWIDGPVLKDLKGSRFDPTAAQKTLWDQWFPFGSNEIGPNGLLKTPKDFKFPLYIIGTGLVDEDLQELSEAGPIDHPQIPDASGILPNSEQVYYGDTGVSKNPIELIEGWFANWKETVMSIVGLVLLITIVFTVLKCIGTCRSLRRKRKIEKDIELQEIGPYQPTTYRPR

SEQ ID NO: 18 - protein, 531 aa, EEL VIRUS EUROPEAN X
MDTLIKILLIIVILKSLHAHIEFVPHDLSKWRDISIEHLDCPIYGDLSNQATETTPVKYSSVQWGLKNNIDGYLCISAKWSVTCDYRWYGSKYISTSIEYVPTKESECRDAIKSSKNGELVSPHFMPENCGWNNVLTESVTYTTVSSHEVKLDPYQMTFVDSLFPGGKCSSSVCSTIYHQGVWINPSNNLGFCKDPVDHQGQLYMAGLVGARGEIVKEVWNLRSVFKPEIGRSKHLTGSCWMTYCDQRGLRFSDGEWAGFQIPEISALKQVLLGLPECKDDVLVHAHDTNTELREILEHMDESALNAICQQEVRRAKERGVVSDWLLSMMTPFTEGLGPVYRLNKGKLEASMGYYRKVYIDSSNAPQAFGQTEDKESVGWSDLVPKDADGAILSMYNGNVVINNQIKWAKNALGSHILDEISALEFETPVVHHPHLTILSVNHSDLVSSTHPNGQGVNLIESVSHWAGGLWASIGSGLMILVLVALVGFCTIKVCLAYVPSIWAQKNRNGKRRGTTSQRSTEQEMFELSAV

SEQ ID NO: 19 - protein, 522 aa, LAGOS BAT VIRUS
MSQLNLIPFFCVIIVLSVEDFPLYTIPEKIGPWTPIDLIHLSCPNNLQSEDEGCGTSSVFSYVELKTGYLTHQKVSGFTCTGVVNEAVTYTNFVGYVTTTFKRKHFKPTALACRDAYHWKISGDPRYEESLHTPYPDNSWLRTVTTTKESLVIISPSIVEMDVYSRTLHSPMFPTGTCSRFYPSSPSCATNHDYTLWLPDDPNLSLACDIFVTSTGKKSMNGSRMCGFTDERGYYRTIKGACKLTLCGKPGLRLFDGTWISFPRPEVTTRCLPNQLVNIHNNRIDEVEHLIVEDLIRKREECLDTLETVLMSKSISFRRLSHFRKLVPGYGKAYTILNGSLMETNVHYLKVDNWSEILPSKGCLKINNQCVAHYKGVFFNGIIKGPDGHILIPEMQSSLLKQHMDLLKAAVFPLKHPLIEPGSLFNKDGDADEFVDVHMPDVHKLVSDVDLGLPDWSLYALIGATIIAFFILICLIRICCKKGGRRNSPTNRPDLPIGLSTTPQPKSKVISSWESYKGTSNV

SEQ ID NO: 20 - protein, 512 aa, SNAKEHEAD RHABDOVIRUS
MTLPNMKPKRIVLFLVFLNAWVSNAQVTHKPRPDSIVEYSEEWENPIYTTPSHCFEDTFAPVKPEKLRCPHIFDDQNLGLTASKAKILHMDLKPEDTHFEAKGRLLHKVTYQVLCSTGFFGGRTVTRKVLETPMGDNEAQAYKAVDREFPYFPEPLCFWLRDNVAAERVFHFSTPKTVTVDLYSRKYISPDFVGGQCAKSPCPTHWPNVYWVGETQSPECPSIDTEGGHIFTKKDTHRITKAVVHGHHPWGLTKACQIQFCNEQWIRTDLGDLIRIEPNDGTSSLTLPKCQDNVVQMRGNLDDFSYLNHAIVNMAQRSECLEAHSSIVAQQKVSPYLLSKFRPPHPGLGKAHYLQNNTIMRGDCIYEGVAEISENRTTYRNLKGEWKKWSLSRGGEGYDGMTVGTKIVIPDLEKYQSIYDNGMFIPKLLGEVPHPSIVITYNQTDSIETGIFTDGKLLNMGVNWTLWPSLSGISLFTVASLILIWYCCCRVTPQALNYSIPMHTITSRGVEI

SEQ ID NO: 21 - protein, 507 aa, VIRAL HEMORAGIC SEPTICEMIA SE-SVA31
MEWNTFFLVILIIIIKNTTPQITQRPPVENISTYHADWDTPLYTHPSNCREDSFVPIRPAQLRCPHEFEDINKGLVSVPTRIIHLPLSVTSVSAVASGHYLHRVTYRVTCSTSFFGGQTIEKTILEAKLSRQEATNEASKDHEYPFFPEPSCIWMKNNVHKDITHYYKTPKTVSVDLYSRKFLNPDFIEGVCTTSPCQTHWQGVYWVGATPTAHCPTSETLEGHLFTRTHDHRVVKAIVAGHHPWGLTMACTVTFCGTEWIKTDLGDLIQVTGPGGARKLTPKKCVNTDIQMRGATDDFSYLNHLITNMAQRTECLDAHSDITASGKISSFLLSKFRPSHPGPGKAHYLLDGQIMRGDCDYEAVVSINYNSAQYKTVNNTWKSWKRVNNNTDGYDGMIFGDKLIIPDIEKYQSVYDSGMLVQRNLVEVPHLSIVFVSNTSDLSTNHIHTNLIPSDWSFNWSLWPSLSGMGVVGGAFLLLVLCCCCKASPPIPNYGIPMQQFSRSQMV

SEQ ID NO: 22 - protein, 526 aa, CARAJAS VIRUS
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWVPYGENSIGPNGLRKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRFLGGLNYL

SEQ ID NO: 23 - protein, 507 aa, VIRAL HEMORAGIC SEPTICEMIA VIRUS FR-L59X; identical to SEQ ID NO: 4

SEQ ID NO: 24 - protein, 561 aa, DROSOPHILA MELANOGASTER SIGMA VIRUS HAP23
MVHYETHILFIHLWMLALIFITTSVWLAASQKTFTPDLVFPEMNRNSSWSVANYGEILCPTSFQSYDPKKHQILTRVLVERPSLNTDTKVEGYTCHKVKYETICDMPWYFSPTISHSISPLRVKESECKDAIAEHQLGTHVSLSFPPEDCSWNSVNTKAYEDIIVKDHPVMLDPYTNNYVDAIFPGGISSPGMGGTIHDDMMWVSKDLAVSPECSGWQQSMGLIYSSRLYGEREPMLEVGSIHIEGHRDKNLTLACRISFCGEIGVRFHDGEWMKVSVNLDHPNSVTFQVTDFPPCPPGTTIQTAVVENINPEIQELTVNMMYRLKCQETISKMVSGLPTSALDLSYLIQVQEGPGIVYKREKGVLYQSVGMYQYIDTVTLNTEENQLGENARGQKVFWTEWSDSPTRPDLQEGINGIVKYEGQIRVPLGMSLRLEAATELMWGHPVHTVSHPILHVISNHTEQSVTTWNRGVNSTNLIGLATRSISGFYNDLKLYLILALIVVSIVALVVLDVIPFKYILFILCPPLLLCRFIKCSRRRPETGDRYHVEYNRPGQVSSAF

SEQ ID NO: 25 - protein, 542 aa, ORCHID FLECK VIRUS
MSTFCSLTVLMFLLISGFEALSLVPKTVCEKEVGLHVDEWLHSCLGACKSSDNDLDMTPHMLMEPTLEYFKAFGYFIHVSTLTKSSHTFLLGGCHITSQETPSDSQELPTNMAENILRHGGPEGEIFMTKEPQCSLWSDNYVKGMLVKYHRVILTVSHTKSGITVLYEQEGVIGHGKVGKTTSSSGTLVWDVRAQYPKCNYRPTGVLSCKREESYIRCRGMSEEKISSITEDCGVIILTTDTRNIYGYHKHDGALTTTQTDDNQIGMVKKIIEIENLMCHHLCESSSEEGGVTHHEYLVSTPIGPWLAVSTESHRTMFMCTDESAVSLVVPVVLCGNGPLVKVRVNEREQWWNISSPYVGMGTHCVPGLSTGLSTVDGVINTWLGQVTIVNGSFTFKRDFINKNFHGAFRPSQFSWSHKTGNDLEHIIDALNTQEKVLSHSHVIESHSVGAGENAVHSFIGMFTTVFEWIQSLIPNVKGWIIKIFLWALLAALVILLLWILWKVLWLFIKSVFLRRTIQAVPTSENSDTSLNRAIHNWAKMD

SEQ ID NO: 26 - protein, 508 aa, SINIPERCA CHUATSI RHABDOVIRUS
MKLIIAPTLISQAIGYPLFVPIRLQGWHDVKLDTLMCPSYASELNKEAAWPQIGLRHLAATDHYEVKGTICHKTTWVKTCDFRWYGPRYITTKISYAPVTGLECQQAIVKASKDELETPYMPEDNCNWATISDNEKTFITVQKSNIFMDPYNMVYVSTVLKGGRCASTVCPLEMHGGIWIPSEAPRESCKLGSSITSHINPNNASRLVTEASYLVTEYRRQLPFLGACRMSMCGEVGMRFKSGEWYKIESSDGRVLSFIASVPMCDGELTVSIHDSSATYHKLSQEILDLSAQIACISELRRAREKKAVSNYLLSFLTPNHGGFGTAYRVLNGQLQASKATYVRVKLGALSTATNWGQLDDGSAYSSEDVTGEIVNGPLFNGNRMDNGTLRVVQNAILGQTLEDEDLYEHSAKEILHPHLTILSSNESDVLSAFRPVGAQGDIIHAVGEWVGTGVGGFIHTIVYLVILCGIIFLLYRCLPYFLKKRKSQSTSQMTPQMIPLQQYQFVP

SEQ ID NO: 27 - protein, 537 aa, CALIGUS ROGERCRESSEYI CROG-EVP-513-004 VIRUS
MKSVFFFFVILFNTMDADLSIPLGNTNNLPGLTTPLEPTVEGRSRTSRKEKDFGEMTVLPLRRLEPWRECAIADLQCPPRYDFGEKIGSLITTEKLWPVRGLSVLQEGYFCTKTSRDRTCSTSFFGSEDLSGSEEYLYPNDSDCLKEVKSLESGRYSPPVWPEHTCAWMATRTVTLVQYQLNLHNVLWEDIGGTYHDAKLKGGKCSTRICSTNNPGVLWIRGKKANHLLRPEDRLPCKIYENKTSSILQVHCDYHHPLHFKVGACKFSYRGESGIRSEEGVGLAWDLKKGSKIARYVGPECDKKKTPIYQWSANSKFRYDAATKADDDLHARCLDALVRIREHKDISQWDLGYFYPSSPGPFPAYRLNKSSKVLECSKYLFTLKEVKSGRSLVDYLPEESIIPDPRTGEKLGVSGLIVKNDSSVYVPHQRSNQMISWEHHLGPKDKVEISRMSLTPNVDDNTLLFSEDPQNHATENGTSQATFLSHLGFGIEHLFYTLLGLSIAGLVGVCVVKVCLENAFKCGCKYCSRSDLDDYSH

SEQ ID NO: 28 - protein, 517 aa, TROUT RHABDOVIRUS903/87
MDLLLKILLIIIIIKEARTRIAFVPLDLGTWRATSVDQLDCPMQGDLVNQGTDAIEVRYQTAAWGLKNSVAGKLCVTAKWSITCDYRWYGSKYISTQIEYLPTTPEMCKEAKRQLDRGELLSPHFSSENCGWNNVLTESETYTTIVSHPVKLDAYSLTLIDNIFEGGKCHEEECPSLLHQGFWIADKNSLGLCKQLDSHIGRLFKTGLRNSMGTVVRQQWDINSIFQPEIGREKNFNGACTMEFCGHQGLRFADREWFKIPEQANQSLKKVFKDLPSCGTDNLVHSHDINTDVKEITEHVHEVALHAICLQEVRRAKDSNTVSDWLLSMMTPFSEGAGPVYRIHNGKLESTVGFYRKVLIEGGGTTEQLGIGFDKKPIKWDQFVVKTNDTRVQSMYNGNTVVDGEIHWAKNVLGSHILDEIAALEFDIPLIQHPHLDGILINETHVISSHHPNGQGVNFVESVTHWAGGIWESVGSSAIIIAVILVVGFVLVKFCMRFSMPVRRERREEGMMMLQRA

SEQ ID NO: 29 - protein, 507 aa, HIRAME RHABDOVIRUS KOREA/CA 9703/1997
DPRIMYYTVLLTTAARVYGQTIKPGVDSVSDQPTWANPLFTYPVDCPAAKLSKVSPSQLRCPRIFDDENRGLVAYPAVIRSLSVGNNLGDIHTQGEYVHKVLYRTTCSTGFFGGQTIEKALVEMKLAPREVGVYDTTTASALYFPAPRCQWYTDNVHNDLTFYYTTAKSVLRDPYTLGFLDSDFIEGKCSKSPCQTHWSNVVWKGDSGVAACDTGSEIKGHIFVDKTSHHVVKATSYGHHPWGLHRACMITFCGKPWIRTDLGDLIAIEYNGGATLLAFPACKDTTVGMRGSLDDFAYLDDLVKSSESREECLEAHAEIIATNSVTPYLLSKFRSPHPGINDVYAMHDGSIYHGKCMTVAIDEVSKDRRTYRAHQTSAFVAWGHPFGDEWGGFHGLHGNDTPVIPDLEKYVAQYKVSMMDKMDIRPVPHPSVQILYNDTDTADITIRKIDSFDLQSLNWSFWPSLSALGGVPILLALVFFLYCCMNRRPSMPAAPQEIPMYHLASRG

SEQ ID NO: 30 - protein, 529 aa, PIRY VIRUS
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 31 - protein, 508 aa, PIKE FRY RHABDOVIRUS
MSIYYALFMVSLMSGCESIPIFVPQNQDISWQPVIRPFNYQCPIHGNLPDTFGLKSTKLTFWSPSVFSPGTVSGWICHAAEWKTTCDYRWYGPQYITHSIHSIRPTIEECKRSIKQLESGTDEDLGFPPQSCGWASVTTVSNKNYKVVVHPVHLEPYQGKWVDHEFLGGECNAPVCEMRGNHSIWLTDHILKEECDQHIEETTGTMYGNARRGDNLYVNNFIIDDHHRVYKFQDACRMKFCGMDGIKFLRGDWIEKKGELARLHENVPDCADGTLISGHKPGADLVDTVFNLENLLEFTLCESTKAKINRREEITSVDLSYLAPRVGGLGSIFRVRNGTLERGSTIYMRIEVEGPIVPRLEGFDPRTNQSRVFWDDWDLDGEVYQGFNGIYKGQDQMIHIPLNMIESGLVDEELQRSFQADTIPHPHFSDDGVSDEDIFFDNTGENGNPVDAVVEWVSGWGTSLKFFGMTLIALVFLFIIIRVVIAVIYCLKKPKKTTVESREMRSLV

SEQ ID NO: 32 - DNA, 33 bp, Artificial Sequence
  PCR primer
cttactagttggaagggctaattcactcccaac

SEQ ID NO: 33 - DNA, 32 bp, Artificial Sequence
  PCR primer
cattctagaactgctagagattttccacactg

SEQ ID NO: 34 - DNA, 42 bp, Artificial Sequence
  PCR primer
taccccgggccatggcctccaaaaaagcctcctcactacttc

SEQ ID NO: 35 - DNA, 44 bp, Artificial Sequence
  PCR primer
actcccgggtaattttttttatttatgcagaggccgaggccgcc

SEQ ID NO: 36 - DNA, 35 bp, Artificial Sequence
  PCR primer
tacacgcgtggagttccgcgttacataacttacgg

SEQ ID NO: 37 - DNA, 39 bp, Artificial Sequence
  PCR primer
cgtggatccgatcgcggtgtcttctatggaggtcaaaac

SEQ ID NO: 38 - DNA, 19 bp, Artificial Sequence
  PCR primer
cccaagaacccaaggaaca

SEQ ID NO: 39 - DNA, 25 bp, Artificial Sequence
  PCR primer
agacaagatagaggaagagcaaaac

SEQ ID NO: 40 - DNA, 27 bp, Artificial Sequence
  Lenti TM probe
aaccattaggagtagcacccaccaagg

SEQ ID NO: 41 - DNA, 24 bp, Artificial Sequence
  PCR primer
cggtgaggatcttcatgaggtagt

SEQ ID NO: 42 - DNA, 20 bp, Artificial Sequence
  PCR primer
aacaccccagccatgtacgt

SEQ ID NO: 43 - DNA, 22 bp, Artificial Sequence
  Humura ACT TM probe
ccagccaggtccagacgcagga